(19) United States
(12) Patent Application Publication
(10) Pub. No.: US 2007/0243595 A1
(43) Pub. Date: Oct. 18, 2007

(54) Modified enzymes, methods to produce modified enzymes and uses thereof

(76) Inventors: Kathleen A. Clarkson, Palo Alto, CA (US); Fred Fenel, Helsinki (FI)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 11/404,460

(22) Filed: Apr. 14, 2006

Related U.S. Application Data
(63) Continuation of application No. 10/565,954, filed on Jul. 31, 2006.

(51) Int. Cl. C12N 9/24; C12N 9/42; C07H 21/04

(52) U.S. Cl. 435/200; 536/23.2; 435/209

(57) ABSTRACT

The invention is directed to modified xylanases having increased stability in harsh industrial environments, such as increased pH and/or temperature.

### FIELD OF THE INVENTION

The invention is directed to modified enzymes having increased stability in harsh industrial environments, such as increased pH and/or temperature.

### BACKGROUND OF THE INVENTION

Xylanases have been found in at least a hundred different organisms. Xylanases are glycosyl hydrolases which hydrolyse β-1,4-linked xylopyranoside chains. Within the sequence-based classification of glycosyl hydrolase families established by Henrissat and Bairoch (1993), most xylanases are found in families 10 and 11. Common features for family 11 members include high genetic homology, a size of about 20 kDa and a double displacement catalytic mechanism (Tenkanen et al., 1992; Wakarchuk et al., 1994). The families have now been grouped, based on structure similarities, into Clans (Henrissat and Davies, 1995). Family 11 glycosyl hydrolases, which are primarily xylanases, reside in Clan C along with family 12 enzymes, all of which are known to be cellulases.

Xylanases can be often used for important applications such as the bleaching of pulp, modification of textile fibers and in animal feed (e.g., xylanases can aid animal digestion, Prade, 1996). Xylanases are useful for production of human foods as well. For example, xylanase improves the properties of bread dough and the quality of bread. Xylanases can also aid the brewing process by improving filterability of xylan containing beers. Xylanases can be employed in the decomposition of vegetative matter including disposal/use of agricultural waste and waste resulting from processing of agricultural products, including production of fuels or other biobased products/materials from biomass.

Often, however, extreme conditions in these applications, such as high temperature and/or pH, etc, render the xylanases less effective than under normal conditions. During pulp bleaching, for example, material that comes from an alkaline wash stage can have a high temperature, sometimes greater than 80° C., and a high pH, such as a pH greater than 10. Since most xylanases do not function well under those conditions, pulp must be cooled and the alkaline pH neutralized before the normal xylanase can function. Taking some of these steps into account, the process can become more expensive since it must be altered to suit the xylanase.

In another example, xylanases are also useful in animal feed applications. There, the enzymes can face high temperature conditions for a short time (e.g. −0.5-5 min at 95° C. or higher) during feed preparation. Inactivation of the enzyme can occur under these temperature conditions, and the enzymes are rendered useless when needed at a lower temperature such as, for example, ˜37° C.

Xylanases with improved qualities have been found. Several thermostable, alkalophilic and acidophilic xylanases have been found and cloned from thermophilic organisms (Bodie et al., 1995; Fukunaga et al., 1998). However, it is often difficult to produce the enzymes in economically efficient quantities. T. reesei, on the other hand, produces xylanases, which are not as thermostable as xylanases from thermophilic organisms. T. reesei is known to produce different xylanases of which xylanases I and II (XynI and XynII, respectively) are the best characterized (Tenkanen et al., 1992). XynI has a size of 19 kDa, a pI of 5.5 and a pH of between 3 and 4. XynII has a size of 20 kDa, a pI of 9.0 and a pH optimum of 5.0-5.5 (Törrönen and Rouvinen, 1995). These xylanases exhibit a favorable pH profile, specificity and specific activity in a number of applications, and can be produced economically in large-scale production processes.

Efforts have been made to engineer a xylanase with favorable qualities. For example, some have tried to improve the stability of the Bacillus circulans xylanase by adding disulphide bridges which bind the N-terminus of the protein to the C-terminus and the N-terminal part of the α-helix to the neighbouring β-strand (Wakarchuk et al., 1994). Also, Campbell et al. (1995) modified Bacillus circulans xylanase by inter- and intramolecular disulphide bonds in order to increase thermostability. Similarly, the stability of T. reesei xylanase II has been improved by changing the N-terminal region to a respective part of a thermophilic xylanase (Sung et al., 1998). In addition to the improved thermostability, the activity range of the enzyme was broadened to include an alkaline pH. Single point mutations have also been used to increase the stability of Bacillus pumilus xylanase (Arase et al., 1993).

By comparing the structures of thermophilic and mesophilic enzymes much information has been obtained (Vogt et al., 1997). Structural analysis of thermophilic xylanases has also given information about factors influencing the thermostability of xylanases (Gruber et al., 1998; Harris et al., 1997).

Currently, however, there is a need for enzymes, especially xylanases, with improved properties in industrial conditions.

### SUMMARY OF THE INVENTION

The current invention relates to modified enzymes. Specifically, the invention relates to modified enzymes with improved performance at extreme conditions of pH and temperature.

In a first aspect, the invention is drawn to a modified xylanase comprising a polypeptide having an amino acid sequence as set forth in SEQ ID NO:1, wherein the sequence has at least one substituted amino acid residue at a position selected from the group consisting of: 2, 5, 7, 10, 11, 16, 19, 22, 26, 28, 29, 30, 34, 36, 38, 57, 58, 61, 63, 65, 67 92, 93, 97, 105, 108, 110, 111, 113, 132, 143, 144, 147, 149, 151, 153, 157, 160, 162, 165, 169, 180, 184, 186, 188, 190 and +191. Preferably, the substitution is selected from the group consisting of: 2, 22, 28, 58, 65, 92, 93, 97, 105, 108, 144, 162, 180, 186 and +191. Preferably, the modified xylanase has at least one substitution selected from the group consisting of: H22K, S65C, N92C, F93W, N97R, V108H, H144C, H144K, F180Q and S186C. Also, preferably, the modified xylanase exhibits improved thermophilicity, alkalophilicity or a combination thereof, in comparison to a wild-type xylanase.

In a second aspect, the invention is drawn to a modified enzyme, the modified enzyme comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H144K, F180Q and S186C.

In a preferred embodiment of the invention, the modified enzyme is a glycosyl hydrolase of Clan C comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H144K, F180Q and S186C. Preferred modified enzymes are as disclosed herein.

In a preferred embodiment, the modified enzyme is a family 11 xylanase comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H 144K, F180Q and S186C. Preferred modified family 11 enzymes are as disclosed herein.

In another preferred embodiment, the modified enzyme is a family 12 cellulase comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H144K, F180Q and S186C, wherein the position is an equivalent position, as defined herein. Preferred family 12 modified enzymes are as disclosed herein.

In a preferred embodiment, the family 12 cellulase is Trichoderma EGIII cellulase as set forth in SEQ ID NO:3, the modification comprises at least one amino acid selected from the group consisting of: 2, 13, 28, 34, 77, 80, 86, 122, 123, 134, 137, 140, 164, 174, 183, 209, 215 and 218, the position numbering being with respect to SEQ ID NO:3. In a preferred embodiment, the substitution is at least one mutation selected from the group consisting of T2C, N13H, S28K, T34C, S77C, P80R, S86C, G122C, K123W, Q134H, Q134K, Q134R, V137H, G140C, N164C, N164K, N174C, K183H, N209C, A215D and N218C, position numbering being with respect to SEQ ID NO:3.

Embodiments of the first and second aspects of the invention, as disclosed above, also provide for nucleic acids encoding any of the modified enzymes, as set forth above, as well as complements. In another preferred embodiment, the invention provides for compositions comprising at least one modified enzyme, as disclosed herein, and another ingredient. In another preferred embodiment, the invention provides vectors comprising a modified enzyme, as disclosed herein, cells comprising the modified enzyme and methods of expressing the modified enzyme.

In a third aspect, the invention is drawn to a method of modifying an enzyme comprising modifying a first site in the enzyme so that the first site can bind to a second site in the enzyme. In a preferred embodiment, the first site is in a loop or sequence adjacent to a β-sheet. In a preferred embodiment, the second site is located in a β-sheet.

In a preferred embodiment, the modified enzyme is a xylanase. For example, in a preferred embodiment, the invention is drawn to a modified xylanase, wherein the xylanase is modified by at least one of the following methods: (i) by modifying an N-terminal sequence so that the N-terminal sequence is bound by a disulphide bridge to an adjacent β-strand; (ii) by modifying a C-terminal sequence so that the C-terminal sequence is bound to an adjacent β-strand; (iii) by modifying an α-helix or sequence adjacent to an α-helix, so that the α-helix, or sequence adjacent to the α-helix, is bound more tightly to the body of the protein; (iv) by modifying a sequence adjacent to the β-strand so that the sequence adjacent to the β-strand can be bound more tightly to an adjacent sequence. For example, in a preferred embodiment, modification can occur in a β-strand next to the cord.

### BRIEF DESCRIPTION OF FIGURES

FIG. 1 shows an amino acid alignment among family 11 xylanases. The amino acid numbering is compared with T. Reesei Xylanase II, as indicated at the top of the sequences. The residues common to at least 75% of family 11 xylanases are underlined. The following are aligned (by abbreviation) in the figure: XYN2_TRIRE Endo-1,4-beta-xylanase 2 precursor (EC 3.2.1.8) (Xylanase 2) (1,4-beta-D-xylan xylanohydrolase 2)—Trichoderma reesei (Hypocrea jecorina)>sp|P36217|; XYN1_TRIRE Endo-1,4-beta-xylanase 1 precursor (EC 3.2.1.8) (Xylanase 1) (1,4-beta-D-xylan xylanohydrolase 1)—Trichoderma reesei (Hypocrea jecorina)>sp|P36218|; XYN2_BACST Endo-1,4-beta-xylanase precursor (EC 3.2.1.8) (Xylanase) (1,4-beta-D-xylan xylanohydrolase)—Bacillus stearothermophilus >sp|P45703|; XYN1_HUMIN Endo-1,4-beta-xylanase 1 precursor (EC 3.2.1.8) (Xylanase 1) (1,4-beta-D-xylan xylanohydrolase 1)—Humicola insolens >sp|P55334|; XYN1_ASPAW Endo-1,4-beta-xylanase I precursor (EC 3.2.1.8) (Xylanase I) (1,4-beta-D-xylan xylanohydrolase I)—Aspergillus awamori >sp|P55328|; XYNA_BACST Endo-1,4-beta-xylanase A precursor (EC 3.2.1.8) (Xylanase A) (1,4-beta-D-xylan >sp|P45705|.

FIG. 2 shows an amino acid alignment of family 12 Cellulases with XynII. The following are aligned (by abbreviation) in the figure: 1ENX XylanaseII Trichoderma reesei, and cel12 family members Q8NJY2 Aspergillus awamori, Q8NJY3 Humicola grisea, Q8NJY4 Trichoderma viride, Q8NJY5 Hypocrea koningii, Q8NJY6 Hypocrea schweinitzii, Q8NJY7 Stachybotrys echinata, Q8NJY8 Bionectria ochroleuca, Q8NJY9 Bionectria ochroleuca, Q8NJZ0 Bionectria ochroleuca, Q8NJZ1 Bionectria ochroleuca, Q8NJZ2 Fusarium solani (subsp. Cucurbitae), Q8NJZ3 Fusarium solani (subsp. cucurbitae), Q8NJZ4 Fusarium equiseti (Fusarium scirpi), Q8NJZ5 Emericella desertorum, Q8NJZ6 Chaetomium brasiliense, Q9KIH1 Streptomyces sp. 11AG8. In the Figure, the two arrows indicates the position of the disulphide bridges (signal sequence not removed).

FIG. 3 shows the nucleotide sequence of the Trichoderma reesei oligonucleotides used in mutagenesis of the xylanase, with the codon changes underlined.

FIG. 4 shows a graph comparing activity with respect to temperature of the wild-type XynII with the Y2 and Y5 mutated xylanases. Mutated xylanases have the following mutations: K58R and an aspartic acid added to the C-terminal serine at position 190 (+191D ) (=Y2); T2C, T28C, K58R+191D, (=Y5). The figure exemplifies that a salt bridge, alone, does not increase thermophilicity and thermal stability. Rather, introduction of a disulphide bridge increases stability and temperature dependent activity. Activity is measured as per Bailey at el., 1992.

FIG. 5 shows a graph comparing the activity with respect to pH of the XynII wild-type with the Y5 mutated xylanase with the following mutations: T2C, T28C, K58R with an added aspartic acid added to the C-terminal serine position 190 (+191D). Activity is measured as per Bailey et al., 1992.

FIG. 6 shows a graph comparing the activity with respect to temperature of the XynII wild-type with the Y5 mutated xylanase with the following mutations: T2C, T28C, K58R with an added aspartic acid added to the C-terminal serine position 190 (+191D). Activity is measured as per Bailey et al., 1992.

FIG. 7 shows a graph comparing the residual activity at pH 5.0, with inactivation at pH 8 with respect to temperature of the wild type XynII xylanase with the Y5 mutated xylanase having the following mutations: T2C, T28C, K58R with an added aspartic acid added to the C-terminal serine position 190 (+191D). Activity is measured as per Bailey et al., 1992.

FIG. 8 shows a graph comparing the residual activity at pH 5.3, with inactivation at pH 8 with respect to temperature of the Y5 mutated xylanase with a XynII xylanase (SS105/162) having the following additional mutations Q162C and L105C. Activity is measured as per Bailey et al., 1992.

FIG. 9 shows a graph comparing the residual activity at pH 5, with inactivation at pH 9 with respect to temperature of the Y5 mutated xylanase with a XynII xylanase (P9) having the following additional mutations: F93W, N97R and H144K. Activity is measured as per Bailey et al., 1992.

FIG. 10 shows a graph comparing the residual activity at pH 5, with inactivation at pH 5 with respect to temperature of the Y5 mutated xylanase with a XynII xylanase (P12) having the following additional mutations H144C and N92C. Activity is measured as per Bailey et al., 1992.

FIG. 11 shows a graph comparing the residual activity at pH 5, with inactivation at pH 9 with respect to temperature of the Y5 mutated xylanase with a XynII xylanase (P12) having the following additional mutations H144C and N92C. Activity is measured as per Bailey et al., 1992.

FIG. 12 shows a graph comparing the residual activity at pH 5.2, with inactivation at pH 8 with respect to temperature of the Y5 mutated xylanase with a XynII (P15) xylanase having the following additional mutations: F180Q, H144C and N92C. Activity is measured as per Bailey et al., 1992.

FIG. 13 shows a graph comparing the residual activity at pH 5, with inactivation at pH 8 with respect to temperature of the Y5 mutated xylanase with a XynII xylanase (P21) having the following additional mutations: H22K, F180Q, H144C and N92C. Activity is measured as per Bailey et al., 1992.

FIG. 14 shows a graph comparing the residual activity at pH 5.17 with inactivation at pH 7.8, with respect to temperature of the Y5 mutated xylanase with a XynII xylanase (P20) having the following additional mutations: H22K and F180Q. Activity is measured as per Bailey et al., 1992.

FIG. 15 shows a graph comparing the activity at pH 8 with respect to temperature of the Y5 mutated xylanase with a XynII xylanase (J17) having the following additional mutation: V108H. Activity is measured as per Bailey et al., 1992.

FIG. 16 shows a graph comparing the activity at pH 8 with respect to temperature of the Y5 mutated xylanase with a XynII xylanase (J21) having the following additional mutations: S65C and S186C (J21 in the graph). Activity is measured as per Bailey et al., 1992.

FIG. 17 shows a structural alignment of Trichoderma reesei xylanaseII (XynII, PDB 1 ENX, in blue;) and Trichoderma reesei endoglucanaseIII (Cal12A, PDB 1H8V, in red).

FIG. 18 sets forth the nucleotide amino acid of sequence of XynII.

FIG. 19 sets forth the nucleotide amino acid of sequence of EGIII.

FIG. 20 sets forth the nucleotide amino acid of sequence of Xyn1I.

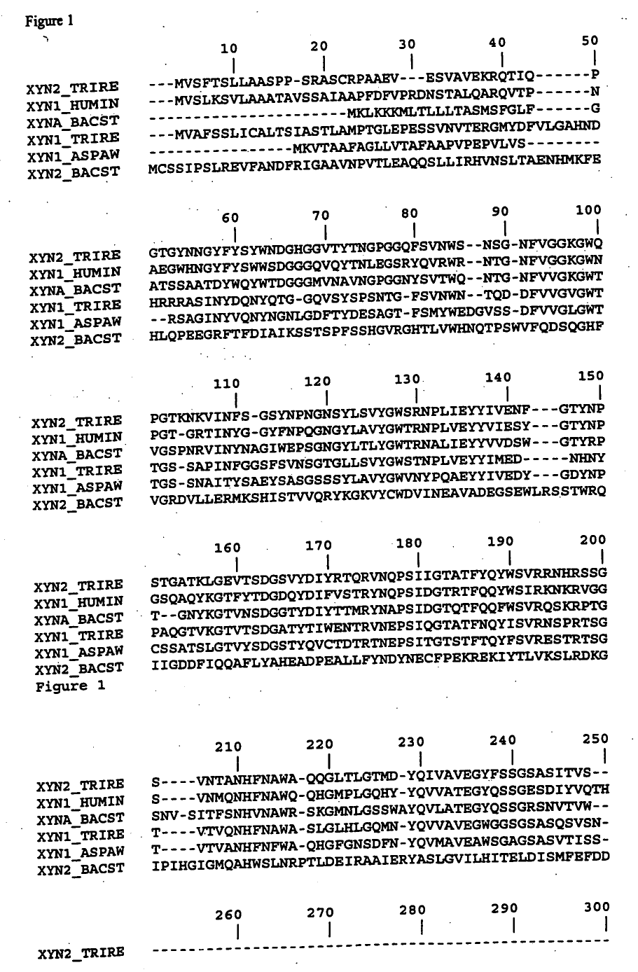

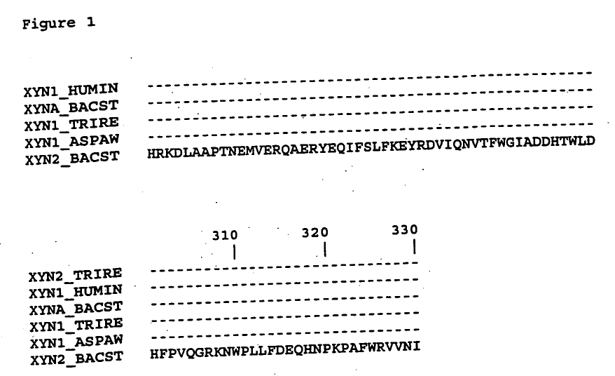

### DETAILED DESCRIPTION OF THE PREFERRED EMBODIMENTS

The invention will now be described in detail by way of reference only using the following definitions and examples. Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

All publications cited herein are expressly incorporated herein by reference for the purpose of describing and disclosing compositions and methodologies which might be used in connection with the invention.

As used herein, the term “polypeptide” refers to a compound made up of a single chain of amino acid residues linked by peptide bonds. The term “protein” herein may be synonymous with the term “polypeptide” or may refer, in addition, to a complex of two or more polypeptides.

As used herein, the term “expression” refers to the process by which a polypeptide is produced based on the nucleic acid sequence of the gene. The process includes both transcription and translation.

As used herein, the term “gene” means the segment of DNA involved in producing a polypeptide chain, that may or may not include regions preceding or following the coding region.

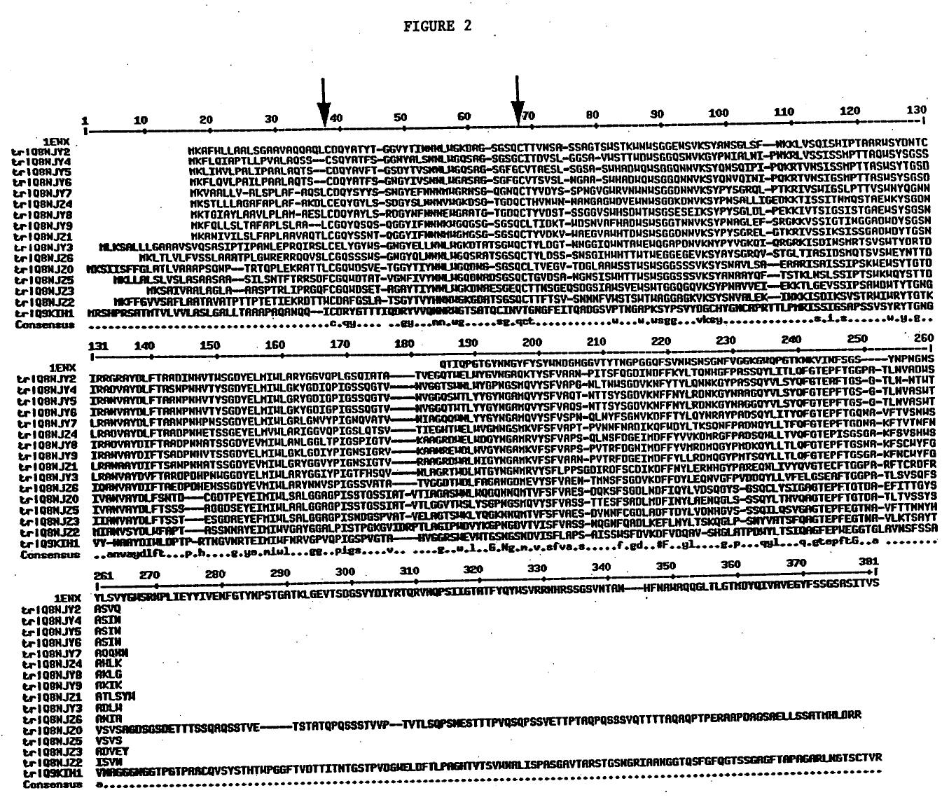

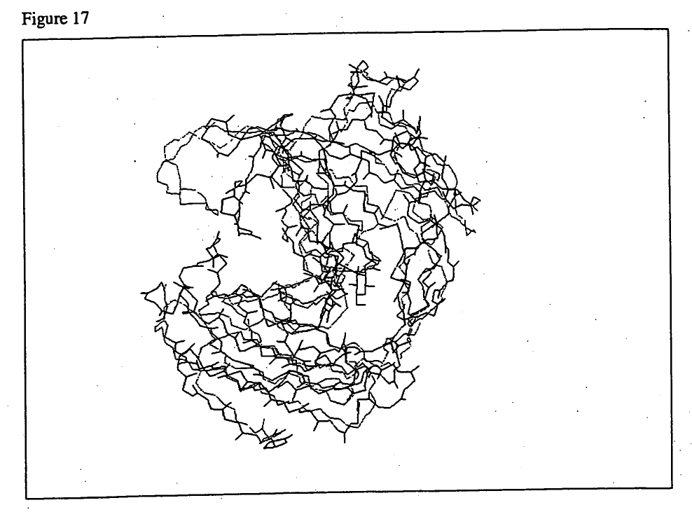

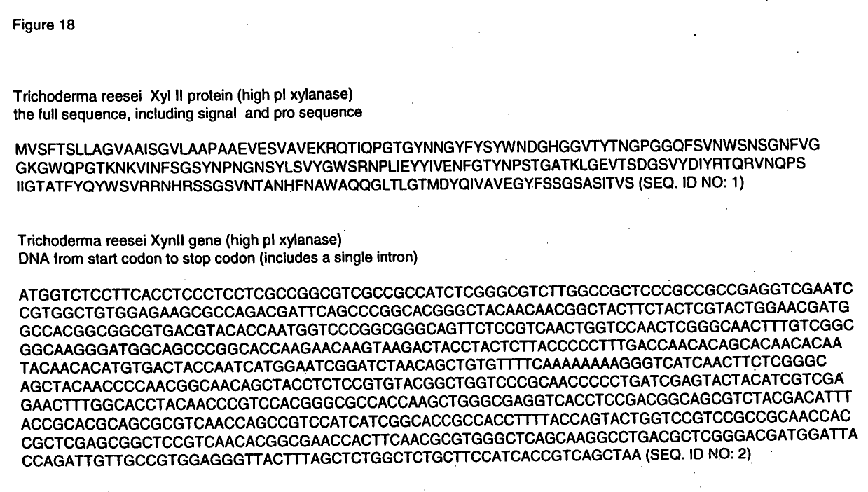

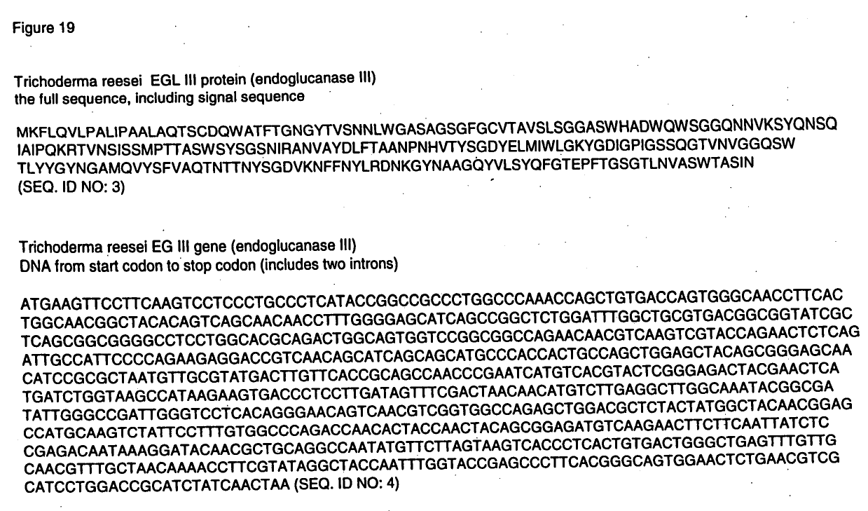

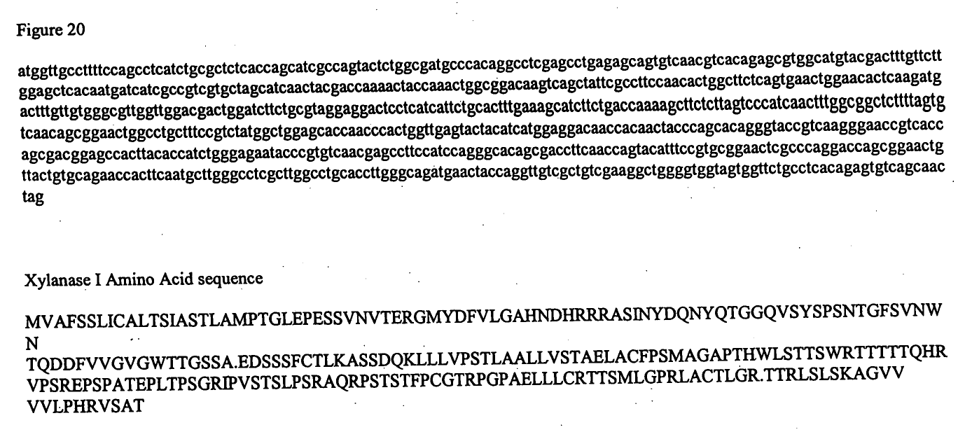

As used herein, when referring to position numbering, the term “equivalent” refers to positions as determined by sequence and structural alignments with Trichoderma reesei xylanase II (xynII) as a reference sequence or reference structure, as provided herein (see, for example, FIG. 2 for a multiple sequence alignment and Trichoderma reesei xylanaseII with other sequences, and FIG. 17 for a structural alignment of Trichoderma reesei Xyn II with Trichoderma reesei endoglucanaseIII). Position numbering shall be with respect to Trichoderma reesei xynII, as set forth in SEQ ID NO:1. The numbering system, even though it may use a specific sequence as a base reference point, is also applicable to all relevant homologous sequences. Sequence homology between proteins may be ascertained using well-known alignment programs and as described herein and by using hybridisation techniques described herein.

As used herein, the term “adjacent” refers to close linear and/or close spatial proximity between amino acid residues or regions or areas of a protein. For example, a first residue or first region or first area which is adjacent to a second residue or second region or second area (in a linear sense), respectively, shall have preferably about 7, preferably about 5, preferably about 2 intervening amino acid residues between them. Alternatively, for example, when a first set of residues or a first region or first area is adjacent to a second set of residues or a second region or second area, then the first set of residues or first region or first area shall be proximal (in space, as shown, for example, by the tertiary structure of a protein) to the second set of residues or second region or second area. One skilled in the art, when possible, would know how to solve the tertiary structure of a protein.

As used herein, when referring to sequence positions, the designation “+” followed by an integer shall mean that a polypeptide has been modified to include additional amino acid(s) at the putative position, as specified by the integer. For example, the designation +191 shall mean that a polypeptide which normally has 190 amino acids in the amino acid sequence has an added amino acid.

As used herein, the term “nucleic acid molecule” includes RNA, DNA and cDNA molecules. It will be understood that as a result of the degeneracy of the genetic code, a multitude of nucleotide sequences encoding a given protein, such as the mutant proteins of the invention, may be produced.

As used herein, the term “disulphide bridge” or “disulphide bond” refers to the bond formed between the sulphur atoms of cysteine residues in a polypeptide or a protein. In this invention, a disulphide bridge or disulphide bond may be non-naturally occurring and introduced by way of point mutation.

As used herein, the term “salt bridge” refers to the bond formed between oppositely charged residues, amino acids in a polypeptide or protein. In this invention, a salt bridge may be non-naturally occurring and introduced by way of point mutation.

As used herein, an “enzyme” refers to a protein or polypeptide that catalyzes a chemical reaction.

As used herein, the term “activity” refers to a biological activity associated with a particular protein, such as enzymatic activity associated with a protease. Biological activity refers to any activity that would normally be attributed to that protein by one skilled in the art.

As used herein, the term “xylanase” refers to glycosyl hydrolases that hydrolyse β-1,4-linked xylopyranoside chains.

As used herein, “XynI” refers to the Trichoderma reesei xylanase, xylanase I. XynI has a size of 19 kDa, a pI of 5.5 and a pH optimum of between 3 and 4.

As used herein, “XynII” refers to the Trichoderma reesei xylanase, xylanase II. XynII has a size of 20 kDa, a pI of 9.0 and a pH optimum of between 5 and 5.5.

As used herein, “xylopyranoside” refers to a β-1,4-linked polymer of xylose, including substituted polymers of xylose, i.e. branched β-D-1,4-linked xylophyranose polymers, highly substituted with acetyl, arabinosyl and uronyl groups (see, for example, Biely, P. (1985) Microbial Xylanolytic Systems. Trends Biotechnol., 3, 286-290.).

As used herein, the term “glycosyl hydrolase” refers to an enzyme which hydrolizes the glycosidic bond between two or more carbohydrates or between a carbohydrate and a non-carbohydrate moiety. Enzymatic hydrolysis of the glycosidic bond takes place via general acid catalysis and requires two critical residues: a proton donor and a nucleophile/base. The IUB-MB Enzyme nomenclature of glycosyl hydrolases is based on substrate specificity and occasionally on molecular mechanism.

As used herein, the term “hydrolase” refers to an enzyme that catalyzes a reaction whereby a chemical bond is enzymatically cleaved with the addition of a water molecule.

As used herein, “hydrolysis” refers to the process of the reaction whereby a chemical bond is cleaved with the addition of a water molecule.

As used herein, “Clan C” refers to groupings of families which share a common three-dimensional fold and identical catalytic machinery (see, for example, Henrissat, B. and Bairoch, A., (1996) Biochem. J.,316, 695-696).

As used herein, “family 11” refers to a family of enzymes as established by Henrissat and Bairoch (1993) Biochem J.,293, 781-788 (see, also, Henrissat and Davies (1997) Current Opinion in Structural Biol. 1997, &:637-644). Common features for family 11 members include high genetic homology, a size of about 20 kDa and a double displacement catalytic mechanism (see Tenkanen et al., 1992; Wakarchuk et al., 1994). The structure of the family 11 xylanases includes two large β-sheets made of β-strands and α-helices. Family 11 xylanases include the following: Aspergillus niger XynA, Aspergillus kawachii XynC, Aspergillus tubigensis XynA, Bacillus circulans XynA, Bacillus pumilus XynA, Bacillus subtilis XynA, Neocallimastix patriciarum XynA, Streptomyces lividans XynB, Streptomyces lividans XynC, Streptomyces thermoviolaceus XynII, Thermomonospora fusca XynA, Trichoderma harzianum Xyn, Trichoderma reesei XynI, Trichoderma reesei XynII, Trichoderma viride Xyn.

As used herein, “family 12” refers to a family of enzymes established by Henrissat and Bairoch (1993) in which known glycosyl hydrolases were classified into families based on amino acid sequence similarities. To date all family 12 enzymes are cellulases. Family 12 enzymes hydrolyze the β-1,4-glycosidic bond in cellulose via a double displacement reaction and a glucosyl-enyzme intermediate that results in retention of the anomeric configuration of the product. Structural studies of family 12 members reveal a compact β-sandwich structure that is curved to create an extensive substrate binding site on the concave face of the β-sheet.

As used herein, the term “protease” refers to an enzyme that degrades by hydrolyzing at least some of their peptide bonds.

As used herein, “peptide bond” refers to the chemical bond between the carbonyl group of one amino acid and the amino group of another amino acid.

As used herein, “wild-type” refers to a sequence or a protein that is native or naturally occurring.

As used herein, “point mutations” refers to a change in a single nucleotide of DNA, especially where that change shall result in a change in a protein.

As used herein, “mutant” refers to a version of an organism or protein where the version is other than wild-type. The change may be affected by methods well known to one skilled in the art, for example, by point mutation in which the resulting protein may be referred to as a mutant.

As used herein, “mutagenesis” refers to the process of affecting a change from a wild-type into a mutant.

As used herein, “substituted” and “modified” are used interchangeably and refer to a sequence, such as an amino acid sequence comprising a polypeptide, that includes a deletion, insertion, replacement or interruption of a naturally occurring sequence. Often in the context of the invention, a substituted sequence shall refer, for example, to the replacement of a naturally occurring residue.

As used herein, “modified enzyme” refers to an enzyme that includes a deletion, insertion, replacement or interruption of a naturally occurring sequence.

As used herein, “β-strands” refers to that portion of an amino acid sequence that forms a linear sequence that occurs in a β-sheets.

As used herein, “β-sheets” refers to the sheet-type structure that results when amino acids hydrogen-bond to each other to form a sheet like structure.

As used herein, “α-helix” refers to the structure that results when a single polypeptide chain turns regularly about itself to make a rigid cylinder in which each peptide bond is regular hydrogen-bonded to other peptide bonds in the nearby chain.

As used herein, “thumb” refers to a loop between β-strands B7 and B8 in XynI and in XynII (see, for example, in Torronen, A. and Rouvinen, J.; Biochemistry 1995, 34, 847-856).

As used herein, “cord” refers to a loop between β-strands B7 and B8 which make a thumb and a part of the loop between β-strands B6a and B9 which crosses the cleft on one side (see, for example, Torronen, A. and Rouvinen, J.; Biochemistry 1995, 34, 847-856).

As used herein, “alkaline” refers to the state or quality of being basic.

As used herein, “alkalophilic” refers to the quality of being more robust in an alkaline atmosphere than a non-alkalophilic member. For example, an alkalophilic organism refers to an organism that survives or thrives under alkaline conditions where a normal organism may not, and an alkalophilic protein is one whose activity is active or more robust under alkaline conditions where a normal protein would be less active.

As used herein, “acidic” refers to the state or quality of being acidic.

As used herein, “acidophilic” refers to the quality of being more robust in an acidic atmosphere than a non-acidophilic member. For example, an acidophilic organism refers to an organism that survives or thrives under acidic conditions where a normal organism may not, and an acidophilic protein is one whose activity is active or more robust under acidic conditions where a normal protein would be less active.

As used herein, “thermostable” refers to the quality of being stable in an atmosphere involving temperature. For example, a thermostable organism is one that is more stable under specified temperature conditions than a non-thermostable organism.

As used herein, “thermostability,” refers to the quality of being thermostable.

As used herein, “thermophilic” refers to the quality of being more robust in an hot atmosphere than a non-thermophilic member. For example, a thermophilic organism refers to an organism that survives or thrives under hot conditions where a normal organism may not, and a thermophilic protein is one whose activity is active or more robust under hot conditions where a normal protein would be less active.

As used herein, “mesophilic” refers to the quality of being more robust in an normal atmosphere than a non-mesophilic member. For example, a mesophilic organism refers to an organism that survives or thrives under normal conditions where another organism may not, and a mesophilic protein is one whose activity is active or more robust under normal conditions where another protein would be less active.

As used herein, “oligonucleotides” refers to a short nucleotide sequence which may be used, for example, as a primer in a reaction used to create mutant proteins.

As used herein, “codon” refers to a sequence of three nucleotides in a DNA or mRNA molecule that represents the instruction for incorporation of a specific amino acid into a polypeptide chain.

As used herein, “Y5” refers to a mutant xylanse as disclosed, for example, in publication number WO 01/27252.

As used herein, the following designations shall refer to the following mutants:

“P2”=N97R+H144K/Y5

“P3”=F93W+H144K in Y5

“P8”=F180Q in Y5

“P9”=N97R in F93W+H144K in Y5

“P12”=H144C+N92C in Y5

“P15”=F180Q in H144C+N92C in Y5

“P16”=N97R in H144C+N92C in Y5

“P18”=H22K in Y5

“P20”=H22K+F180Q in Y5

“P21”=H22K+F180Q+H144C+N92C in Y5

“J17”=V108H in Y5

“J21”=S65C+S186C in Y5

wherein position numbering shall be with respect to XynII.

The present invention relates to modified enzymes with improved performance in extreme conditions, such as temperature and pH.

In a first aspect, the invention is drawn to a modified xylanase comprising a polypeptide having an amino acid sequence as set forth in SEQ ID NO:1, wherein the sequence has at least one substituted amino acid residue at a position selected from the group consisting of: 2, 5, 10, 11, 16, 19, 22, 26, 28, 29, 30, 34, 36, 38, 57, 58, 61, 63, 65, 67 92, 93, 97, 105, 108, 110, 111, 113, 132, 143, 144, 147, 149, 151, 153, 157, 160, 162, 165, 169, 180, 184, 186, 188, 190 and +191, where position numbering is with respect to SEQ ID NO:1. Preferably, the substitution is selected from the group consisting of: 2, 22, 28, 58, 65, 92, 93, 97, 105, 108, 144, 162, 180, 186 and +191. Preferably, the modified xylanase has at least one substitution selected from the group consisting of H22K, S65C, N92C, F93W, N97R, V108H, H144C, H144K, F180Q and S186C. Also, preferably, the modified xylanase exhibits improved thermophilicity, alkalophilicity or a combination thereof, in comparison to a wild-type xylanase.

In a second aspect, the invention is drawn to a modified enzyme, the modified enzyme comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 44, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191, wherein position numbering is with respect to SEQ ID NO:1. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H144K, F180Q and S186C.

In a preferred embodiment of the invention, the modified enzyme is a glycosyl hydrolase of Clan C comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 110, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, is S65C, N92C, F93W, N97R, V108H, H144C, H144K, F180Q and S186C. Preferred modified enzymes are as disclosed herein.

In a preferred embodiment, the modified enzyme is a family 11 xylanase comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H144K, F180Q and S186C. Preferred modified family 11 enzymes are as disclosed herein.

In another preferred embodiment, the modified enzyme is a family 12 cellulase comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191. In a preferred embodiment, the amino acid sequence has at least one substituted amino acid residue at a position equivalent to a position selected from the group consisting of: 2, 22, 28, 58, 65, 92, 93, 97, 105, 108, 144, 162, 180, 186 and +191. In a preferred embodiment, the amino acid sequence has at least one substituted amino acid residue selected from the group consisting of: H22K, S65C, N92C, F93W, N97R, V108H, H144C, H144K, F180Q and S186C. Preferred family 12 modified enzymes are as disclosed herein.

In a preferred embodiment, the family 12 cellulase is Trichoderma EGIII cellulase as set forth in SEQ ID NO:3, the modification comprises at least one amino acid selected from the group consisting of: 2, 13, 28, 34, 77, 80, 86, 122, 123, 134, 137, 140, 164, 174, 183, 209, 215 and 218, position numbering being with respect to SEQ ID NO:3. In a preferred embodiment, the substitution is at least one mutation selected from the group consisting of T2C, N13H, S28K, T34C, S77C, P80R, S86C, G122C, K123W, Q134H, Q134K, Q134R, V137H, G140C, N164C, N164K, N174C, K183H, N209C, A215D and N218C, position numbering being with respect to SEQ ID NO:3.

XynII exhibits a significant amino acid homology with other members of family 11, approximately 20-90%, as well as overall structural similarity. Homology, as used herein, may be determined by one skilled in the art; specifically, homologies of at least 20%, preferably 30% or more, preferably 40% or more, preferably 50% or more, preferably 60% or more, preferably 70% or more, preferably 80% or more, preferably 90% or more, preferably 95% or more and preferably 97% or more are contemplated (as calculated at the amino acid level and the nucleotide level and as used herein). There are structural similarities between family 11 and family 12 enzymes. Beta proteins have two stacked beta sheets, and one alpha helix packed against one of the beta sheets forming a so-called beta-jelly roll structure. (see Stirk, H. J., Woolfson, D. N., Hutchison, E. G. and Thornton, J. M. (1992) Depicting topology and handedness in jellyroll structures. FEBS Letters 308 p 1-3).

Based on this structural similarity, both enzyme families have been assigned to a “super family” referred to as Clan C (see Sandgren, M. et. al., J. Mol. Bio. (2001) 308, 295-310.)).

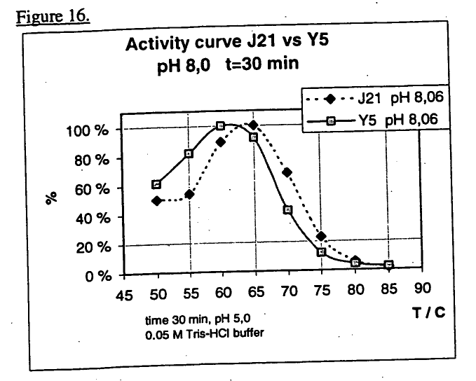

Although the sequence homology between families 11 and 12 is low, the overall structural similarity of the two families is remarkable as seen by comparing FIGS. 2 and 16. The length of the loops connecting the two beta-sheets comprises the major structural differences between the families (Sandgren et. al., J. Mol., Biol., 2001). Presently, no family 11 enzymes are known to contain N terminal disulphide bridges while many family 12 cellulases, in general appear to contain a disulphide bridge near the N-terminus (e.g, between residues 4 and 32 in T. reesei Cel 12A). That disulphide bridge in family 12 enzymes is located near the position where a disulphide was introduced into the Trichoderma (Y5) variant, although further away from the N-terminus (see, for example, publication WO 01/27252). The importance of a restriction stabilizing the N-terminal region of family 11 enzymes was examined in Trichoderma reesei xylanase II (XynII). By inserting a non-natural disulphide bridge between residues (T2C and T28C), an increase in Tₘ of 11° C. was achieved. In these two structurally similar families, family 11 and family 12, the N-terminal disulphide bridges play a similar roles regarding stability. This has been demonstrated by replacing the cysteine at position 32 with an alanine in Cel12A resulting in a significant decrease in Tₘ of 18.5° C. Interestingly, the magnitude of the change in stability for adding a non-natural N-terminal disulphide into XynII is comparable to that of removing a natural one from Cel 12A (see table A).

TABLE A

| Enzyme | Delta Tm | Tm (degrees C.) |
|---|---|---|
| WT Cel12A | | 54.4 |
| C32A | −18.5 | 35.9 |
| WT xynII | | 58.6 |
| Y5 | +10.7 | 69.3 |

Table A shows the melting temperatures, Tₘ of the wild type Cel12A compared to the variant with the substitution at position 32, and the wild type XynII compared to the Y5 variant of this enzyme.

The three dimensional structures of the N-terminal disulphide bridges of the three publicly known structures for family 12 glycosyl hydrolases (Trichoderma reesei-PDB 1H8V, Aspergillus niger-PDB 1KS5, Streptomyces lividans-PDB 2NLR), show a shift in the position of the disulphide bridge as compared to the non-natural disulphide bridge at sites 2 and 28 in Y5 xylanase. Table B shows the position of the disulphide bridge in a Y5 xylanase (“PDB 1ENX” being wild type XynII xylanase) and in the three known family 12 structures. The structural positions of the mutations at 2 and 28 of Y5 xylanase can be translated to the corresponding residues in the Cel 12 structures. In each case, the non-native disulphide from Y5 is closer to the N-terminus; and for the A. niger structure (PDB 1KS5) a disulphide could be designed that would utilize the N-terminal residue itself (at residues Q1C, V35C, according to A. niger numbering). Instead of being limited by the natural sequence, X-ray data could be used to design extensions and truncations of the N-terminus to facilitate non-native disulphides that specifically attach to the new N-terminal residues.

TABLE B

| | | | Where (according to |
|---|---|---|---|
| | WT N- | Corresponding | structure) could a |
| | terminal S-S | site to 2-28 | S-S be inserted at |
| Code | position | of xynII | the N-terminal |
| PDB 1ENX | No | — | |
| Y5 | C2-C28 | T2-T28 | T2C-T28C |
| PDB 1H8V | C4-C32 | T2-T34 | T2C-T34C |
| PDB 1KS5 | C4-C32 | T2-Y34 | Q1C-V35C |
| PDB 2NLR | C5-C31 | T3-T33 | T3C-T33C |

A large number of family 12 sequences (Table C) are known which could potentially be stabilized through an N-terminal disulphide bridge, particularly those molecules where a non-native disulphide bridge could be introduced or a native disulphide could be moved closer to an N-terminus. Table C lists a number of sequences where a predicted removal of the signal sequence produces mature protein sequences very similar to the ones of the known family 12 structures. Table C also lists the distance between the two N-terminal cysteines (26-28 amino acids) similar to the disulphide bond of Y5. In the cleavage site predictions, a signal sequences is theoretically removed by the means of known, acknowledged parameters (see, for example, “Identification of prokaryotic and eukaryotic signal peptides and prediction of their cleavage sites”. Henrik Nielsen, Jacob Engelbrecht, Søren Brunak and Gunnar von Heijne, Protein Engineering 10, 1-6 (1997)).

A large group of sequences of unknown three dimensional structures in Table C fall within the structurally similar group of family 12 enzymes, which have in a similar manner a cysteine residue at the N-terminal at site 5±2 residues, forming a disulphide bridge with residue 32±7, such that the first beta strand or strands of the beta sheet can be bound to the adjacent beta sheet. All of these sequences could be treated in the manner described in the discussion around table B to improve stability.

TABLE C

| | | | | Number of | |
|---|---|---|---|---|---|
| | | | | adequate | |
| | | Eucaryote/ | Predicated | cysteine | aa's to 2^{nd} |
| | | Gram−/ | cleavage | (1^{st} in ss | cysteine in |
| ID | Sequence | Gram+ | site | bond) | ss bond |
| Q8NJY2 | Endoglucanase | Eu | 16-17 | 6 | 28 |
| | {GENE:CEL12B} | | | | |
| | Aspergillus awamori | | | | |
| | (var. kawachi) | | | | |
| Q8NJY4 | Endoglucanase | Eu | 16-17 | 4 | 28 |
| | {GENE:CEL12A} - | | | | |
| | Trichoderma viride | | | | |
| Q8NJY5 | Endoglucanase | Eu | 16-17 | 4 | 28 |
| | {GENE:CEL12A} - | | | | |
| | Hypocrea koningii | | | | |
| Q8NJY6 | Endoglucanase | Eu | 16-17 | 4 | 28 |
| | {GENE:CEL12A} - | | | | |
| | Hypocrea schweinitzii | | | | |
| Q8NJY7 | Endoglucanase | Eu | 16-17 | 4 | 28 |
| | {GENE:CEL12A} - | | | | |
| | Stachybotrys echinata | | | | |
| Q8NJY8 | Endoglucanase | Eu | 17-18 | 4 | 28 |
| | {GENE:CEL12D} - | | | | |
| | Bionectria ochroleuca | | | | |
| Q8NJY9 | Endoglucanase | Eu | 17-18 | 3 | 28 |
| | {GENE:CEL12C} - | | | | |
| | Bionectria ochroleuca | | | | |
| Q8NJZ1 | Endoglucanase | Eu | 18-19 | 4 | 28 |
| | {GENE:CEL12A} - | | | | |
| | Bionectria ochroleuca | | | | |
| Q8NJZ4 | Endoglucanase | Eu | 17-18 | 4 | 28 |
| | {GENE:CEL12A} - | | | | |
| | Fusarium equiseti | | | | |
| | (Fusarium scirpi) | | | | |
| Q9KIH1 | Cellulase12A | Gram+ | 31-32 | 5 | 26 |
| | {GENE:CEL12A} - | | | | |
| | Streptomyces sp. | | | | |
| | 11AG8 | | | | |

Table D lists further a number of sequences of family 12 enzymes with uncleaved signal sequence. They all have cysteines 30-39 amino acids apart, and after a removal of the signal sequence (removal can be performed as in table C) are structurally capable of forming a disulphide bridge at the N-terminal (as seen in the publicly known structures, see table B). The proposed mutation site correlates to the corresponding site of the disulphide bridge between sites 2-28 of the Y5 mutant. The glycosyl hydrolase sequences were aligned using the program MOE (Chemical Computing Corp) using standard sequence matching methods.

TABLE D

| Sequence code | enzyme | Species | Mutations |
|---|---|---|---|
| Tr O94218 | Cel12 | Aspergillus aculeatus | D22C/G52C |
| Sp P22669 | Cel12 | Aspergillus aculeatus | Q20C/T52C |
| Sp Q12679 | Cel12 | Aspergillus awamori | T18C/Y50C |
| Tr O13454 | Cel12 | Aspergillus oryzae | E18C/Y50C |
| Sp P16630 | Cel12 | Erwina carotovora | A32C/I68C |
| Tr O31030 | Cel12 | Pectobacterium carotovora | A32C/V68C |
| Tr Q9V2TO | Cel12 | Pyrococcus furiosus | P57C/T96C |
| Tr O33897 | Cel12 | Rhodothermus marinus | E40C/E70C |
| Tr Q9RJY3 | Cel12 | Streptomyces coelicolor | T43C/T73C |
| Tr O08468 | Cel12 | Streptomyces halstedii | L40C/T70C |
| Tr Q59963 | Cel12 | Streptomyces rochei | T40C/T70C |
| Tr Q9KIH1 | Cel12 | Streptomyces sp. 11AG8 | Q34C/N64C |
| Tr Q60032 | Cel12 | Thermotoga maritima | V2C/K38C |
| Tr Q60033 | Cel12 | Thermotoga maritime | V20C/K56C |
| Tr O08428 | Cel12 | Thermotoga neopolitana | V2C/R38C |
| Tr P96492 | Cel12 | Thermotoga neopolitana | V20C/K56C |
| AF435072 | Cel12A | Aspergillus Kawachi | Q20C/T52C |
| AF434180 | Cel12A | Chaetium brasilience | S28C/Y61C |
| AF434181 | Cel12A | Emericella desertorum | D30C/G63C |
| AF434182 | Cel12A | Fusarium equiseti | D19C/H51C |
| AF434183 | Cel12A | Nectria ipomoeae | Q25C/T58C |
| AF434184 | Cel12B | Nectria ipomoeae | T32C/T65C |
| AF435063 | Cel12A | Bionectria ochroleuca | T20C/Y52C |
| AF435064 | Cel12B | Bionectria ochroleuca | T34C/T66C |
| AF435065 | Cel12C | Bionectria ochroleuca | A18C/T50C |
| AF435066 | Cel12D | Bionectria ochroleuca | S19C/Y51C |
| AF435071 | Cel12A | Humicola grisea | S34C/Y67C |
| AF435068 | Cel12A | Hypochrea schweinitzii | T18C/T50C |
| AF435067 | Cel12A | Stachybotrys echinata | S18C/Y50C |

Not only does the N-terminal region show high structural similarity between families 11 and 12; both families show a hand like structure, the one of a “partly closed right hand” as described in Törrönen et al. 1997. The two β-sheets form “fingers”, and a twisted pair from one β-sheet and the a-helix forms a “palm”. The long loop between β-strands B7 and B8 makes the “thumb” and a part of the loop between β-strands B6b (residues 95-102 in xynII and 125-131 in Cel12A) and B9 forms a “cord”, which crosses the cleft on one side (Torronen A. and Rouvinen, J. Biochem. 1995, 34, 847-0856). The stabilizing effect of inserting rigidifying substitutions between beta strand B6b and the adjacent loop and/or the “cord” is seen in the mutation at sites 92, 93, 144 (N92C-H144C, at least one of the following mutations N97R, F93W+H144K (XynII), and can in a similar way be introduced into corresponding sites in family 12.

Table E shows the numbering of a selection of structurally equivalent sites between xynII and Cel 12A. The high structural similarity between the two families enables a large number of similar substitutions (see Sandgren et. al., J. Mol., Biol., 2001 for structural comparison).

TABLE E
Examples of equivalent sites

| XynII | Cel12A |
|---|---|
| T2C | T2C |
| T28C | T34C |
| N92C | G122C |
| H144C, K | N164C, K |
| F93W | K123W |
| Q162H | K183H |

The modified enzymes of the invention may comprise one or more mutations in addition to those set out above. Other mutations, such as deletions, insertions, substitutions, transversions, transitions and inversions, at one or more other locations, may also be included. Likewise, the modified enzyme may be missing at least one of the substitutions set forth above.

The modified enzyme may also comprise a conservative substitution that may occur as a like-for-like substitution (e.g., basic for basic, acidic for acidic, polar for polar etc.) Non-conservative substitutions may also occur, i.e. from one class of residue to another or alternatively involving the inclusion of unnatural amino acids such as ornithine, diaminobutyric acid ornithine, norleucine ornithine, pyriylalanine, thienylalanine, naphthylalanine and phenylglycine.

The sequences may also have deletions, insertions or substitutions of amino acid residues that produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids can be grouped together based on the properties of their side chain alone. However it is more useful to include mutation data as well. The sets of amino acids thus derived are likely to be conserved for structural reasons. These sets can be described in the form of a Venn diagram (Livingstone C. D. and Barton G. J. (1993) “Protein sequence alignments: a strategy for the hierarchical analysis of residue conservation” Comput.Appl Biosci. 9: 745-756)(Taylor W. R. (1986) “The classification of amino acid conservation” J.Theor.Biol. 119; 205-218). Conservative substitutions may be made, for example according to the table below which describes a generally accepted Venn diagram grouping of amino acids.

| Set | | Sub-set | |
|---|---|---|---|
| Hydrophobic | FWYHKMILVAGC | Aromatic | FWYH |
| | | Aliphatic | ILV |
| Polar | WYHKREDCSTNQ | Charged | HKRED |
| | | Positively | HKR |
| | | charged | |
| | | Negatively | ED |
| | | charged | |
| Small | VCAGSPTND | Tiny | AGS |

Variant amino acid sequences may also include suitable spacer groups inserted between any two amino acid residues of the sequence including alkyl groups such as methyl, ethyl or propyl groups in addition to amino acid spacers such as glycine or β-alanine residues. A further form of variation involves the presence of one or more amino acid residues in peptoid form.

Homology comparisons can be conducted by eye, or more usually, with the aid of readily available sequence comparison programs. These commercially available computer s programs can calculate % homology between two or more sequences. % homology may be calculated over contiguous sequences, i.e. one sequence is aligned with the other sequence and each amino acid in one sequence is directly compared with the corresponding amino acid in the other sequence one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p 387). Examples of other software than can perform sequence comparisons include, but are not limited to, the BLAST package (see Ausubel et al., 1999 Short Protocols in Molecular Biology, 4^{th} Ed—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Embodiments of the first and second aspects of the invention, as disclosed above, provide a nucleic acid encoding any of the modified enzymes, as set forth above, as well as complements thereof. In another preferred embodiment, the invention provides for compositions comprising at least one modified enzyme, as disclosed herein, and another ingredient. In another preferred embodiment, the invention provides vectors comprising a modified enzyme, as disclosed herein, cells comprising the modified enzyme and methods of expressing the modified enzyme.

One skilled in the art will be aware of the relationship between nucleic acid sequence and polypeptide sequence, in particular, the genetic code and the degeneracy of this code, and will be able to construct such modified enzymes without difficulty. For example, one skilled in the art will be aware that for each amino acid substitution in the s modified enzyme sequence there may be one or more codons which encode the substitute amino acid. Accordingly, it will be evident that, depending on the degeneracy of the genetic code with respect to that particular amino acid residue, one or more modified enzyme nucleic acid sequences may be generated corresponding to that modified enzyme polypeptide sequence.

Mutations in amino acid sequence and nucleic acid sequence may be made by any of a number of techniques, as known in the art. In particularly preferred embodiments, the mutations are introduced into parent sequences by means of PCR (polymerase chain reaction) using appropriate primers, as illustrated in the Examples. The parent enzymes may be modified at the amino acid level or the nucleic acid level to generate the modified enzyme sequences described herein. Therefore, a preferred embodiment provides for the generation of modified enzymes by introducing one or more corresponding codon changes in the nucleotide sequence encoding a modified enzyme.

It will be appreciated that the above codon changes can be made in any modified enzyme nucleic acid sequence. For example, sequence changes can be made to any of the homologous sequences described herein.

The modified enzyme may comprise the “complete” enzyme, i.e., in its entire length as it occurs in nature (or as mutated), or it may comprise a truncated form thereof. The modified enzyme derived from such may accordingly be so truncated, or be “full-length”. The truncation may be at the N-terminal end or the C-terminal end. The modified enzyme may lack one or more portions, such as sub-sequences, signal sequences, domains or moieties, whether active or not.

A nucleotide sequence encoding either an enzyme which has the specific properties as defined herein or an enzyme which is suitable for modification, such as a modified enzyme, may be identified and/or isolated and/or purified from any cell or organism producing said enzyme. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme or a part of the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library and then plating the transformed bacteria onto agar plates containing a substrate for enzyme thereby allowing clones expressing the enzyme to be identified.

In a yet further alternative, the nucleotide sequence encoding the modified enzyme may be prepared synthetically by established standard methods, e.g. the phosphoroamidite method described by Beucage S. L. et al., (1981) Tetrahedron Letters 22, p 1859-1869 or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin or mixed genomic and cDNA origin, prepared by ligating fragments of synthetic, genomic or cDNA origin in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

The nucleotide sequences described here, and suitable for use in the methods and compositions described here may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of this document, it is to be understood that the nucleotide sequences described herein may be modified by any method available in the art. Such modifications may be carried out in order to enhance the in vivo activity or life span-of nucleotide sequences.

A preferred embodiment of the invention provides for nucleotide sequences and the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the modified enzyme sequences may be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other species and probing such libraries with probes comprising all or part of any one of the sequences in the attached sequence listings under conditions of medium to high stringency. Similar considerations apply to obtaining species homologues and allelic variants of the polypeptide or nucleotide sequences described here.

Variants and strain/species homologues may also be obtained using degenerate PCR which will use primers designed to target sequences within the variants and homologues encoding conserved amino acid sequences. The primers used in degenerate PCR will contain one or more degenerate positions and will be used at stringency conditions lower than those used for cloning sequences with single sequence primers against known sequences. Conserved sequences can be predicted, for example, by aligning the amino acid sequences from several variants/homologues. Sequence alignments can be performed using computer software known in the art as described herein.

Alternatively, such polynucleotides may be obtained by site directed mutagenesis of characterised sequences, as provided herein. This may be useful where, for example, silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides may be used to produce a primer, e.g. a PCR primer, a primer for an alternative amplification reaction, a probe e.g. labelled with a revealing label by conventional means using radioactive or non-radioactive labels or the polynucleotides may be cloned into vectors. Such primers, probes and other fragments will be at least 15, preferably at least 20, for example at least 25, 30 or 40 nucleotides in length, and are also encompassed by the term polynucleotides.

Polynucleotides such as DNA polynucleotides and probes may be produced recombinantly, synthetically or by any means available to those of skill in the art. They may also be cloned by standard techniques. In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector. Preferably, the variant sequences are at least as biologically active as the sequences presented herein.

A preferred embodiment of the invention includes sequences that are complementary to the modified enzyme or sequences that are capable of hybridising either to the nucleotide sequences of the modified enzymes (including complementary sequences of those presented herein), as well as nucleotide sequences that are complementary to sequences that can hybridise to the nucleotide sequences of the modified enzymes (including complementary sequences of those presented herein). A preferred embodiment provides polynucleotide sequences that are capable of hybridising to the nucleotide sequences presented herein under conditions of intermediate to maximal stringency.

A preferred embodiment includes nucleotide sequences that can hybridise to the nucleotide sequence of the modified enzyme nucleic acid, or the complement thereof, under stringent conditions (e.g. 50° C. and 0.2×SSC). More preferably, the nucleotide sequences can hybridise to the nucleotide sequence of the modified enzyme, or the complement thereof, under high stringent conditions (e.g. 65° C. and 0.1×SSC).

It may be desirable to mutate the sequence in order to prepare a modified enzyme. Accordingly, a mutant may be prepared from the modified enzymes provided herein. Mutations may be introduced using synthetic oligonucleotides. These oligonucleotides contain nucleotide sequences flanking the desired mutation sites. A suitable method is disclosed in Morinaga et al., (Biotechnology (1984) 2, p 646-649). Another method of introducing mutations into enzyme-encoding nucleotide sequences is described in Nelson and Long (Analytical Biochemistry (1989), 180, p 147-151). A further method is described in Sarkar and Sommer (Biotechniques (1990), 8, p 404-407—“The megaprimer method of site directed mutagenesis”). Other methods to mutate the sequence are employed and disclosed herein.

In a preferred embodiment, the sequence for use in the methods and compositions described here is a recombinant sequence—i.e. a sequence that has been prepared using recombinant DNA techniques. Such techniques are explained, for example, in the literature, for example, J. Sambrook, E. F. Fritsch, and. T. Maniatis, 1989, Molecular Cloning: A Laboratory Manual, Second Edition, Books 1-3, Cold Spring Harbor Laboratory Press.

Another embodiment provides for compositions and formulations comprising modified enzymes. The compositions include the modified enzyme together with another component.

Another embodiment provides vectors comprising the modified enzyme, cells comprising the modified enzyme and methods of expressing the modified enzyme. The nucleotide sequence for use in the methods and compositions described herein may be incorporated into a recombinant replicable vector. The vector may be used to replicate and express the nucleotide sequence, in enzyme form, in and/or from a compatible host cell. Expression may be controlled using control sequences, e.g., regulatory sequences. The enzyme produced by a host recombinant cell by expression of the nucleotide sequence may be secreted or may be contained intracellularly depending on the sequence and/or the vector used. The coding sequences may be designed with signal sequences which direct secretion of the substance coding sequences through a particular prokaryotic or eukaryotic cell membrane. Polynucleotides can be incorporated into a recombinant replicable vector. The vector may be used to replicate the nucleic acid in a compatible host cell. The vector comprising the polynucleotide sequence may be transformed into a suitable host cell. Suitable hosts may include bacterial, yeast, insect and fungal cells.

Modified enzymes and their polynucleotides may be expressed by introducing a polynucleotide into a replicable vector, introducing the vector into a compatible host cell and growing the host cell under conditions which bring about replication of the vector. The vector may be recovered from the host cell.

The modified enzyme nucleic acid may be operatively linked to transcriptional and translational regulatory elements active in a host cell of interest. The modified enzyme nucleic acid may also encode a fusion protein comprising signal sequences such as, for example, those derived from the glucoamylase gene from Schwanniomyces occidentalis, α-factor mating type gene from Saccharomyces cerevisiae and the TAKA-amylase from Aspergillus oryzae. Alternatively, the modified enzyme nucleic acid may encode a fusion protein comprising a membrane binding domain.

The modified enzyme may be expressed at the desired levels in a host organism using an expression vector. An expression vector comprising a modified enzyme nucleic acid can be any vector capable of expressing the gene encoding the modified enzyme nucleic acid in the selected host organism, and the choice of vector will depend on the host cell into which it is to be introduced. Thus, the vector can be an autonomously replicating vector, i.e. a vector that exists as an episomal entity, the replication of which is independent of chromosomal replication, such as, for example, a plasmid, a bacteriophage or an episomal element, a minichromosome or an artificial chromosome. Alternatively, the vector may be one which, when introduced into a host cell, is integrated into the host cell genome and replicated together with the chromosome.

The expression vector typically includes the components of a cloning vector, such as, for example, an element that permits autonomous replication of the vector in the selected host organism and one or more phenotypically detectable markers for selection purposes. The expression vector normally comprises control nucleotide sequences encoding a promoter, operator, ribosome binding site, translation initiation signal and optionally, a repressor gene or one or more activator genes. Additionally, the expression vector may comprise a sequence coding for an amino acid sequence capable of targeting the modified enzyme to a host cell organelle such as a peroxisome or to a particular host cell compartment. Such a targeting sequence includes but is not limited to the sequence SKL. For expression under the direction of control sequences, the nucleic acid sequence the modified enzyme is operably linked to the control sequences in proper manner with respect to expression.

Preferably, a polynucleotide in a vector is operably linked to a control sequence that is capable of providing for the expression of the coding sequence by the host cell, i.e. the vector is an expression vector. The control sequences may be modified, for example, by the addition of further transcriptional regulatory elements to make the level of transcription directed by the control sequences more responsive to transcriptional modulators. The control sequences may in particular comprise promoters.

In the vector, the nucleic acid sequence encoding for the modified enzyme is operably combined with a suitable promoter sequence. The promoter can be any DNA sequence having transcription activity in the host organism of choice and can be derived from genes that are homologous or heterologous to the host organism. Examples of suitable promoters for directing the transcription of the modified nucleotide sequence, such as modified enzyme nucleic acids, in a bacterial host include the promoter of the lac operon of E. coli, the Streptomyces coelicolor agarase gene dagA promoters, the promoters of the Bacillus licheniformis α-amylase gene (amyL), the aprE promoter of Bacillus subtilis, the promoters of the Bacillus stearothermophilus maltogenic amylase gene (amyM), the promoters of the Bacillus amyloliquefaciens α-amylase gene (amyQ), the promoters of the Bacillus subtilis xylA and xylB genes and a promoter derived from a Lactococcus sp.—derived promoter including the P170 promoter. When the gene encoding the modified enzyme is expressed in a bacterial species such as E. coli, a suitable promoter can be selected, for example, from a bacteriophage promoter including a T7 promoter and a phage lambda promoter. For transcription in a fungal species, examples of useful promoters are those derived from the genes encoding the, Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, Aspergillus oryzae alkaline protease, Aspergillus oryzae triose phosphate isomerase or Aspergillus nidulans acetamidase. Examples of suitable promoters for the expression in a yeast species include but are not limited to the Gal 1 and Gal 10 promoters of Saccharomyces cerevisiae and the Pichia pastoris AOX1 or AOX2 promoters.

Examples of suitable bacterial host organisms are gram positive bacterial species such as Bacillaceae including Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus lautus, Bacillus megaterium and Bacillus thuringiensis, Streptomyces species such as Streptomyces murinus, lactic acid bacterial species including Lactococcus spp. such as Lactococcus lactis, Lactobacillus spp. including Lactobacillus reuteri, Leuconostoc spp., Pediococcus spp. and Streptococcus spp. Alternatively, strains of a gram-negative bacterial species belonging to Enterobacteriaceae including E. coli, or to Pseudomonadaceae can be selected as the host organism. A suitable yeast host organism can be selected from the biotechnologically relevant yeasts species such as but not limited to yeast species such as Pichia sp., Hansenula sp or Kluyveromyces, Yarrowinia species or a species of Saccharomyces including Saccharomyces cerevisiae or a species belonging to Schizosaccharomyce such as, for example, S. Pombe species. Preferably a strain of the methylotrophic yeast species Pichia pastoris is used as the host organism. Preferably the host organism is a Hansenula species. Suitable host organisms among filamentous fungi include species of Aspergillus, e.g. Aspergillus niger, Aspergillus oryzae, Aspergillus tubigensis, Aspergillus awamori or Aspergillus nidulans. Alternatively, strains of a Fusarium species, e.g. Fusarium oxysporum or of a Rhizomucor species such as Rhizomucor miehei can be used as the host organism. Other suitable strains include Thermomyces and Mucor species.

Host cells comprising polynucleotides may be used to express polypeptides, such as the modified enzymes disclosed herein, fragments, homologues, variants or derivatives thereof. Host cells may be cultured under suitable conditions which allow expression of the proteins. Expression of the polypeptides may be constitutive such that they are continually produced, or inducible, requiring a stimulus to initiate expression. In the case of inducible expression, protein production can be initiated when required by, for example, addition of an inducer substance to the culture medium, for example dexamethasone or IPTG. Polypeptides can be extracted from host cells by a variety of techniques known in the art, including enzymatic, chemical and/or osmotic lysis and physical disruption. Polypeptides may also be produced recombinantly in an in vitro cell-free system, such as the TnT™ (Promega) rabbit reticulocyte system.

In a third aspect, the invention is drawn to a method of modifying an enzyme comprising modifying a first site in the enzyme part of a structurally defined region so that the first site can bind to a second site. In a preferred embodiment, the first site is in a loop or sequence adjacent to a β-sheet. In a preferred embodiment, the second site is located in a β-sheet. In a preferred embodiment, the modified enzyme is a xylanase or Clan C.

In a preferred embodiment, the invention is drawn to a modified xylanase or a method of modifying a xylanase (or modified enzyme), according to at least one of the following: (i) modifying the N-terminal sequence so that the N-terminal region is bound by a disulphide bridge to an adjacent β-strand (see Gruber, et al., 1998in T. reesei XynII the amino acids 1-4 and 24-30 respectively); (ii) modifying the C-terminal (in T. reesei XynII amino acids 183-190, see Gruber, et al., 1998) so that it is bound to an adjacent β-strand; (iii) modifying an α-helix of the enzyme so that it can be bound more tightly to the body of the protein; (iv) modifying at least one adjacent loop so that it binds adjacent beta strand B6a (in T. reesei XynII amino acids 91-94, Gruber, et al., 1998) or (v) modifying residue equivalent to XynII, as provided above.

As another embodiment, (per the examples) mutagenesis may be used to create disulphide bridges, salt bridges and separate point mutations at different regions. For example, the enzyme may be modified to create at least one disulphide bridge, so that at least one disulphide bridge may: 1) stabilize the N-terminal region or bind the N-terminal beta strand to the adjacent beta sheet (positions 2-28, 5-19, 7-16, 10-29 in XynII, or an equivalent position, as disclosed herein); 2) stabilize the alpha helix region (positions 105-162, 57-153, 110-151, 111-151, in XynII, or an equivalent position as disclosed herein); 3) stabilize the C-terminal region (positions 63-188, 61-190, 36-186 or 34-188 in XynII, or an equivalent position as disclosed herein); or 4) stabilize the loop by binding to the beta strand such as B6b (92-144, 113-143 in XynII or an equivalent position as disclosed herein) and/or 5) stabilize the beta sheet (positions 26-38, 61-149, 63-147, 65-186, 67-184 in XynII, or an equivalent position, as provided herein).

Salt bridges may be created at different sites of the enzyme: (e.g., positions 22, 180, 58 or +191D in XynII, or an equivalent position, as provided herein) and single point mutations may be introduced at different sites of the molecule (e.g., positions 108, 26, 30, 67, 93, 97, 132, 157, 160, 165, 169 or 186 in XynII, or an equivalent position, as provided herein) thereby increasing the thermostability and/or thermophilicity and or alkalophilicity the protein. As with the Y5 mutant, the C-terminus may be bound more tightly to the body of the enzyme by adding as a recombinant change one amino acid (e.g. aspartic acid or glutamic acid) which then can form a salt bridge from the C-terminus to the body of the enzyme. If appropriate, a suitable amino acid replacement can be made in the body of the protein, so as to enable the formation of a salt bridge or to stabilize the enzyme in the C-terminal part via the α-helix or a region near the a-helix.

Additional mutants can be created according to this aspect of the invention. The structure of the N-terminal beta strand A1 or N-terminal loop in family 11 and 12 enzymes is described as the beta strand, a part of the beta sheet A prior to/up to a beta bend structure leading to beta strand B1 or the N-terminal loop prior to the first beta strand of the beta sheet. (see, Törrönen et al., Biochemistry 1995, 34, 847-856; Sandgren, et. al., J. Mol. Bio. (2001) 308, 295-310; Gruber, et al., 1998). The B1 beta strand of the N-terminal region is described as the beta strand part of the beta sheet B prior to/up to a beta bend structure leading to beta strand B2 or the loop prior to the first beta strand of the beta sheet. The beta strand A1 region is bound preferably to beta strand A2 or to any other adjacent region (XynII or an equivalent thereof). The beta strand B1 region is bound preferably to beta strand B2 or to any other adjacent region (XynII or an equivalent thereof). In XynII A1 comprises residues 1-4, A2 residues 25-30, B1 residues 6-10 and B2 residues 13-19.

The structure of the C-terminal beta strand A4 or C-terminal loop in family 11 and 12 enzymes is the beta strand part of the beta sheet A between beta strands A3 and A5 or the loop as following beta sheet A4 (see Törrönen et al., Biochemistry 1995, 34, 847-856; Sandgren, et. al., J. Mol. Bio. (2001) 308, 295-310; Gruber, et al., 1998). The beta strand A4 region is bound preferably to beta strand A3 or A5, or to any other adjacent region. In XynII A4 is residues 183-190, A3 is residues 33-39 and A5 is residues 61-69. The cord of family 11 and 12 is described as the loop connecting beta strands B6b and B9. The beta strand of family 11 and 12 B6b is described as the beta strand prior to the cord (Törrönen et al., Biochemistry 1995, 34, 847-856; Sandgren, et. al., J. Mol. Bio. (2001) 308, 295-310; Gruber, et al., 1998). The beta strand B6b region may be bound to the cord or to the loop between beta strands A6 and B7, or to any other adjacent region. In XynII, B6b is residues 90-94 and B9 is residues 103-110, the cord is 95-102, beta strand A6 is residues 148-152, beta strand B7 is residues 134-142 and the loop between beta stands A6 and B7 is residues 143-147.

The helix of family 11 and 12 enzymes is described as region following beta strand A6 and forming a helical structure parallel to beta strand B9 (Törrönen et al., Biochemistry 1995, 34, 847-856; Sandgren, et. al., J. Mol.). The helix of family 11 and 12 enzymes is bound preferably to beta strand B9 or any other adjacent region. In XynII the helix is residues 153-162, beta strand A6 is residues 148-152 and beta strand B9 is residues 103-110.

### EXAMPLES

### Example 1

### Plasmids Used for Xylanase II Expression and Mutagenesis Template

The open reading frame encoding Trichoderma reesei XYNII gene product was amplified by polymerase chain reaction (PCR) from the T. reesei cDNA library. XYNII cDNA was cloned into pKKtac (VTT, Espoo, Finland) or alternatively into pALK143 (ROAL, Rajamäki, Finland).

### Example 2

### Site-Directed Mutagenesis for Generation of Mutant of Xylanase II

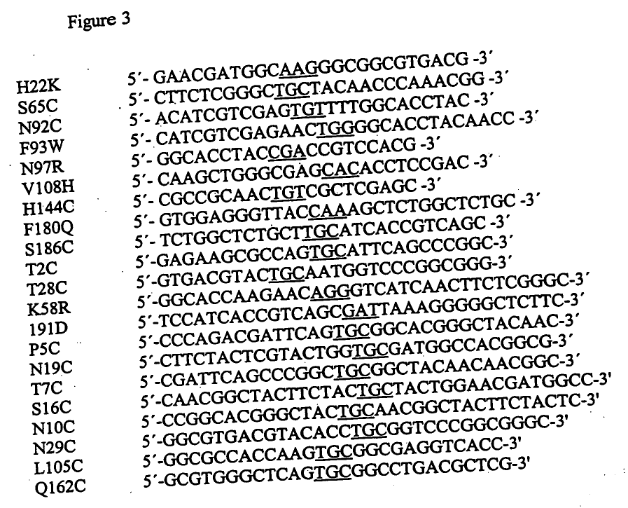

Expression vectors containing cDNA-encoding xylanase II as described in Example 1 were used as template in the stepwise site-directed mutagenesis in consecutive PCR amplifications. Synthetic oligonucleotide primers containing the altered codons for the mutations X-Y were used for insertion of the desired alteration into the native xylanase II primary amino acid sequence. By this approach the residues of sites 92, 93 and 144 of the wild-type enzyme mutants were generated to bind the loop N143-S146 of xynII to the neighbouring β-strand. Additionally, mutagenesis was performed to generate the mutations at sites 22, 65, 97 and 108 into the xylanase primary sequence. The oligonucleotide sequences used in the mutagenesis are shown FIG. 3. PCR was carried out as described in the Quick Change Site-directed mutagenesis (Stratagene, La Jolla, Calif., USA) according to standard PCR procedures. Pfu Turbo (Stratagene) was used as DNA polymerase to amplify plasmid DNA. Plasmid DNA from the site-directed mutagenesis PCR amplification was transformed to E. coli XL-1 blue and the transformed bacterial cells were then propagated on LB, with ampicillin 100 ug/ml for plasmid DNA selection and amplification of the mutated DNA. Plasmids were isolated and sequenced to confirm that they contain the desired mutations. The mutated plasmid DNA encoding the mutant variants was over-expressed in E. coli to examine the influence of the mutagenesis on the T. reesei xylanase Y5 mutants enzymatic properties.

### Example 3

### Production of the Modified XYNII Gene Products in E. coli Strain and Assay for Xylanase Activity

E. coli strains over-expressing the mutated variants of the xylanase II were cultivated on plates supplemented with 1% birchwood xylan (Sigma, Steinheim, Germany) coupled with Rhemazol Brilliant Blue. Rhemazol Brilliant Blue coupled to xylan was utilized to detect xylanase activity that was readily visualized by a characteristic halo formation due to the blue colour disappearance around the bacterial colonies expressing xylanase activity (Biely et al., 1985).

The mutated xylanase genes (see above; Example 2) were expressed in E. coli at +37° C. in shake flasks in LB culture medium. Cell cultures expressing the enzyme variants were centrifuged and the cell pellet separated from the supernatant harbouring the enzyme that was secreted from the cells into the culture medium. The xylanase enzyme activity assay was performed according to standard methods. The growth medium containing the secreted xylanase mutants were incubated for 10 min in 1% birchwood xylan (Sigma) at 50° C. in 50 mM citrate-phosphate buffer (ph 5.0-t) and 50 mM Tris-HCl at pH 7-9. (Bailey et al., 1992). If needed, heat inactivated growth medium was used to dilute the samples. The enzymatic activity of the mutant variants was examined in comparison to the wild type and the Y5 mutation enzyme at varying conditions (see, for Bailey et al., 1992).

### Example 4

### Determination of the Temperature Dependent Stability and pH Dependent Activity of the Xylanase II Mutants

Activity as a Function of Temperature;

The xylanase activity of the mutant variants was determined at varying temperatures and selected pH values (see Figures herein). The mutants were incubated for 10 min with 1% birchwood xylan (Sigma) in 50 mM citrate-phosphate buffer (ph 4.5-7) or 50 mM Tris-HCl at pH 7-9. The relative amount of released reducing sugars was detected with the DNS method assay as described in example 3.

Residual Activity

The mutant variants were incubated for 10 minutes at varying temperatures without substrate. After the inactivation, the samples were cooled on ice and the residual activity was determined by DNS-method as described in example 3.

pH Dependent Activity

The pH-dependent xylanase activity was determined by detecting the enzyme activity at varying pH ranging from XX-YY for 10 min in 1% birchwood xylan at selected temperatures (see pictures) in 50 mM citrate-phosphate buffer (ph 4.5-7) and 50 mM Tris-HCl at pH 7.5-9. This was followed by the DNS assay as described in example 3.

### Example 5

Preparation and Testing of Mutant Xylanases for Improved Properties

Mutant xylanases were prepared having substitutions at one or more substitutions at different regions of the molecule. The substitutions were either separate point mutations in contact with other separate point mutations or they were prepared to act on a structural element found commonly in both family 11 and family 12 enzymes. The enzyme assays were performed as outlined in the examples. Examples of “structural” substitutions are disclosed herein and shown in the examples.

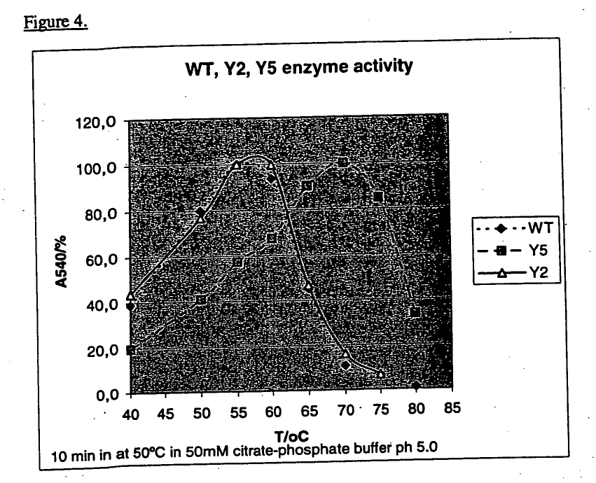

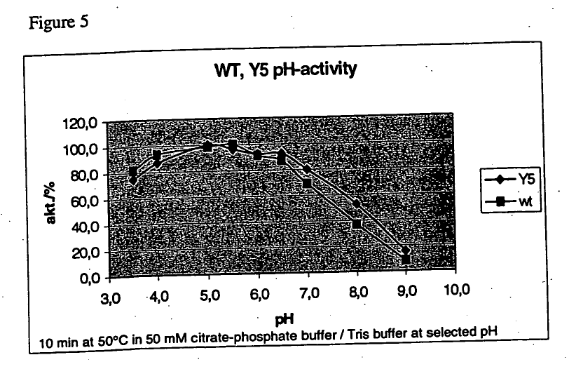

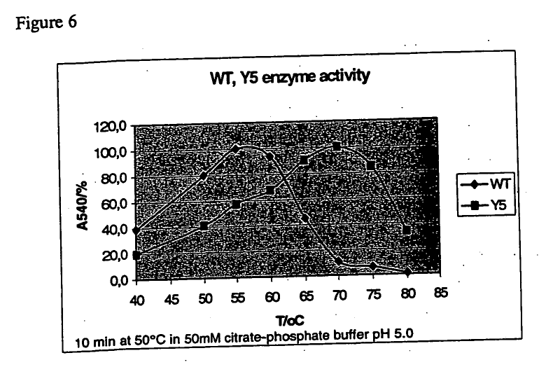

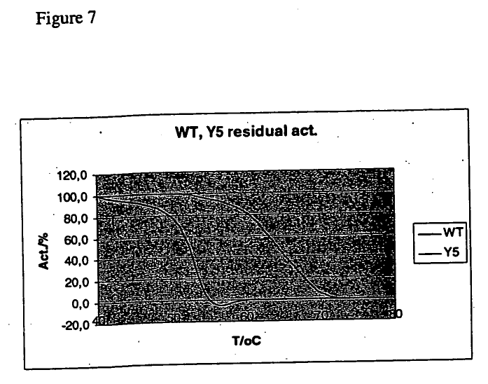

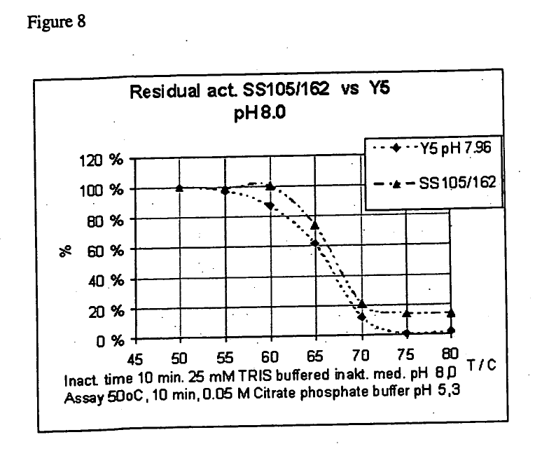

The disulphide bridge can be placed between sites 2 and 28 (T2C, T28C). FIG. 4 shows the importance of the N-terminal region in substituting residues of the wt for a more thermophilic variant. In a similar way removal of the native disulphide bridge (residues C4 and C32, Cel12A numbering) of T. reesei EGIII affects greatly the stability of the enzyme, as shown in the figures provided and tables herein (see, especially, Table A).

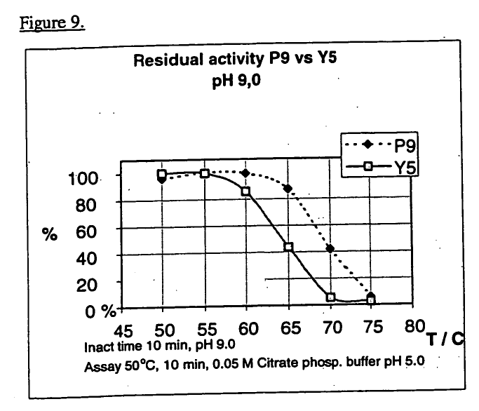

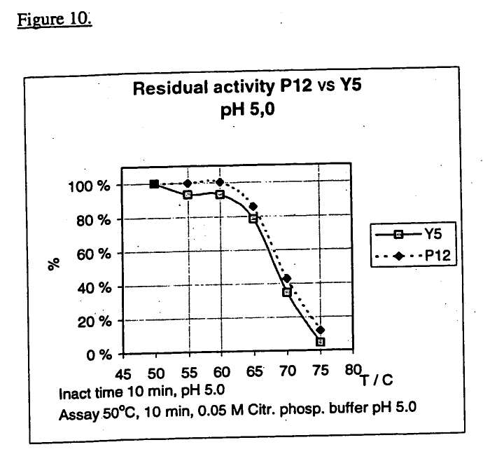

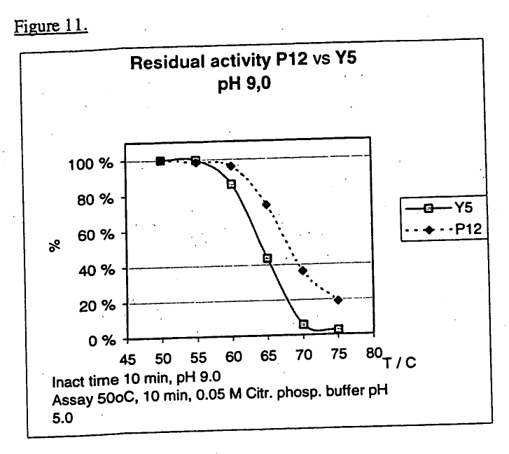

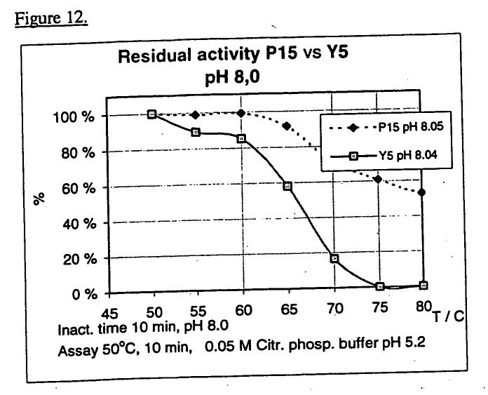

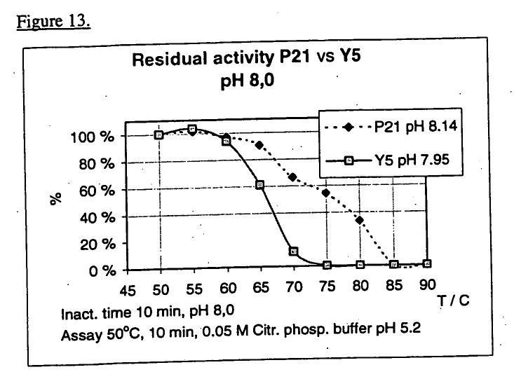

The region of the beta sheet common to both family 11 and 12 named beta strand B6b (as in Gruber et al), is shown to be of importance for stability, especially at alkali conditions. This effect is seen in the substitutions (as compared to the Y5 variant) as improved stability at pH 9 vs pH5 for P12, as shown in the figures (see, for example, FIG. 9, FIG. 10 and FIG. 11).

The importance of the region is clearly demonstrated by a different set of mutations (although in the same region) affecting the same beta strand. When sites 93, 97 and 144 are substituted (F93W, N97R, H144K, P9 in the graph), a similar effect in stabilization of the enzyme as when substituting the sites 92 and 144 (N92C, H144C=P12 in the graph) can be seen in the FIG. 9.

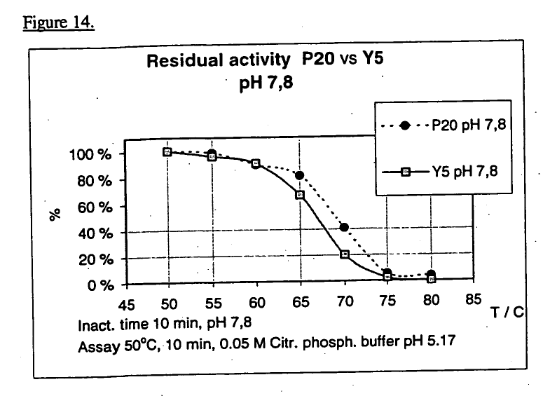

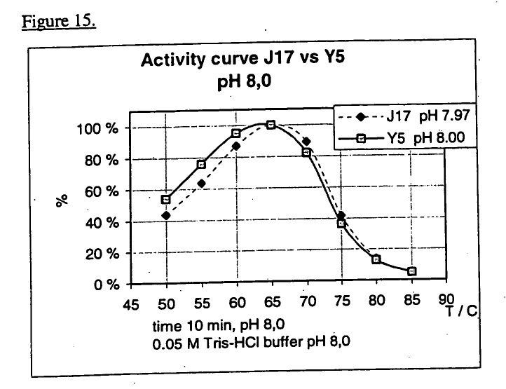

An example of the improved characteristics of separate substitutions at sites 22 and 180 is seen below. The variant containing the substitutions H22K and F180Q (P20 in FIG. 14) shows enhanced thermal stability over Y5 at pH 7.8.

Also the C-terminal region is of important for stability. In the substitution S65C, S186C (J21 in the graph) the enzyme shows improved activity with respect to temperature at pH 8.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A nucleic acid encoding a modified xylanase comprising a polypeptide having an amino acid sequence as set forth in SEQ ID NO:1, wherein the sequence has at least one substituted amino acid residue at a position selected from the group consisting of: 2, 5, 7, 10, 11, 16, 19, 22, 26, 28, 29, 30, 34, 36, 38, 57, 58, 61, 63, 65, 67 92, 93, 97, 105, 108, 110, 111, 113, 132, 143, 144, 147, 149, 151, 153, 157, 160, 162, 165, 169, 180, 184, 186, 188, 190 and +191.

2. The nucleic acid according to claim 1, wherein the substitution is selected from the group consisting of: 2, 22, 28, 58, 65, 92, 93, 97, 105, 108, 144, 162, 180, 186 and +191.

3. The nucleic acid according to claim 2, wherein the xylanase has at least one substitution selected from the group consisting of: H22K, S65C, N92C, F93W, N97R, V108H, H144C, H144K, F180Q and S186C.

4. The nucleic acid according to claim 3, wherein the xylanase has the following mutations: F93W, N97R and H144K.

5. The nucleic acid according to claim 3, wherein the xylanase has the following mutations: H144C and N92K.

6. The nucleic acid according to claim 3, wherein the xylanase has the following mutations: F180Q, H144C and N92C.

7. The nucleic acid according to claim 3, wherein the xylanase has the following mutations: H22K and F180Q.

8. The nucleic acid according to claim 3, wherein the xylanase has the following mutations: V108H.

9. The nucleic acid according to claim 3, wherein the xylanase has the following mutations: S65C and S186C.

10. The nucleic acid according to claim 3, wherein the xylanase has the following mutations: H22K, F180Q, H144C and N92C.

11. A modified xylanase comprising a polypeptide having an amino acid sequence as set forth in SEQ ID NO:1, wherein the sequence has at least one substituted amino acid residue at a position selected from the group consisting of: 2, 5, 7, 10, 11, 16, 19, 22, 26, 28, 29, 30, 34, 36, 38, 57, 58, 61, 63, 65, 67 92, 93, 97, 105, 108, 110, 111, 113, 132, 143, 144, 147, 149, 151, 153, 157, 160, 162, 165, 169, 180, 184, 186, 188, 190 and +191.

12. The xylanase according to claim 11, wherein the substitution is selected from the group consisting of: 2, 22, 28, 58, 65, 92, 93, 97, 105, 108, 144, 162, 180, 186 and +191.

13. The xylanase according to claim 12, wherein the modified xylanase has at least one substitution selected from the group consisting of: H22K, S65C, N92C, F93W, N97R, V108H, H144C, H144K, F180Q and S186C.

14. The xylanase according to claim 13, wherein the xylanase has the following mutations: F93W, N97R and H144K.

15. The xylanase according to claim 13, wherein the xylanase has the following mutations: H144C and N92K.

16. The xylanase according to claim 13, wherein the xylanase has the following mutations: F180Q, H144C and N92C.

17. The xylanase according to claim 13, wherein the xylanase has the following mutations: H22K and F180Q.

18. The xylanase according to claim 13, wherein the xylanase has the following mutations: V108H.

19. The xylanase according to claim 13, wherein the xylanase has the following mutations: S65C and S186C.

20. The xylanase according to claim 13, wherein the xylanase has the following mutations: H22K, F180Q, H144C and N92C.

21. A modified enzyme, the modified enzyme comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191.

22. The enzyme according to claim 21, wherein homology to the sequence set forth in SEQ ID NO:1 is at least 20%.

23. The enzyme according to claim 22, wherein the amino acid sequence has at least one substituted amino acid residue at a position equivalent to a position selected from the group consisting of: 2, 22, 28, 58, 65, 92, 93, 97, 105, 108, 144, 162, 180, 186 and +191.

24. A glycosyl hydrolase of Clan C comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191.

25. The glycosyl hydrolase according to claim 24, wherein homology to the sequence set forth in SEQ ID NO:1 is at least 20%.

26. The glycosyl hydrolase according to claim 25, wherein the amino acid sequence has at least one substituted amino acid residue at a position equivalent to a position selected from the group consisting of: 2, 22, 28, 58, 65, 92, 93, 97, 105, 108, 144, 162, 180, 186 and +191.

27. A modified family 11 xylanase comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191.

28. The xylanase according to claim 27, wherein homology to the sequence set forth in SEQ ID NO:1 is at least 20%.

29. The xylanase according to claim 28, wherein the amino acid sequence has at least one substituted amino acid residue at a position equivalent to a position selected from the group consisting of: 2, 22, 28, 58, 65, 92, 93, 97, 105, 108, 144, 162, 180, 186 and +191.

30. A family 12 cellulase comprising an amino acid sequence, the amino acid sequence being homologous to the sequence set forth in SEQ ID NO:1, the amino acid sequence having at least one substituted amino acid residue at a position equivalent to a position selected from the group consisting of: 2, 5, 7, 10, 11, 16, 19, 22, 26, 28, 29, 30, 34, 36, 38, 57, 58, 61, 63, 65, 67, 92, 93, 97, 105, 108, 110, 111, 113, 132, 143, 144, 147, 149, 151, 153, 157, 160, 162, 165, 169, 180, 184, 186, 188, 190 and +191.

31. The cellulose according to claim 30, wherein homology to the sequence set forth in SEQ ID NO:1 is at least 20%.

32. The cellulose according to claim 31, wherein the amino acid sequence has at least one substituted amino acid residue at a position equivalent to a position selected from the group consisting of: 2, 22, 28, 58, 65, 92, 93, 97, 105, 108, 144, 162, 180, 186 and +191.


SEQUENCE LISTING (51 sequences)

SEQ ID NO: 1 - protein, 223 aa, Trichoderma reesei
MVSFTSLLAGVAAISGVLAAPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 2 - DNA, 781 bp, Trichoderma reesei
atggtctccttcacctccctcctcgccggcgtcgccgccatctcgggcgtcttggccgctcccgccgccgaggtcgaatccgtggctgtggagaagcgccagacgattcagcccggcacgggctacaacaacggctacttctactcgtactggaacgatggccacggcggcgtgacgtacaccaatggtcccggcgggcagttctccgtcaactggtccaactcgggcaactttgtcggcggcaagggatggcagcccggcaccaagaacaagtaagactacctactcttaccccctttgaccaacacagcacaacacaatacaacacatgtgactaccaatcatggaatcggatctaacagctgtgttttcaaaaaaaagggtcatcaacttctcgggcagctacaaccccaacggcaacagctacctctccgtgtacggctggtcccgcaaccccctgatcgagtactacatcgtcgagaactttggcacctacaacccgtccacgggcgccaccaagctgggcgaggtcacctccgacggcagcgtctacgacatttaccgcacgcagcgcgtcaaccagccgtccatcatcggcaccgccaccttttaccagtactggtccgtccgccgcaaccaccgctcgagcggctccgtcaacacggcgaaccacttcaacgcgtgggctcagcaaggcctgacgctcgggacgatggattaccagattgttgccgtggagggttactttagctctggctctgcttccatcaccgtcagctaa

SEQ ID NO: 3 - protein, 234 aa, Trichoderma reesei
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 4 - DNA, 826 bp, Trichoderma reesei
atgaagttccttcaagtcctccctgccctcataccggccgccctggcccaaaccagctgtgaccagtgggcaaccttcactggcaacggctacacagtcagcaacaacctttggggagcatcagccggctctggatttggctgcgtgacggcggtatcgctcagcggcggggcctcctggcacgcagactggcagtggtccggcggccagaacaacgtcaagtcgtaccagaactctcagattgccattccccagaagaggaccgtcaacagcatcagcagcatgcccaccactgccagctggagctacagcgggagcaacatccgcgctaatgttgcgtatgacttgttcaccgcagccaacccgaatcatgtcacgtactcgggagactacgaactcatgatctggtaagccataagaagtgaccctccttgatagtttcgactaacaacatgtcttgaggcttggcaaatacggcgatattgggccgattgggtcctcacagggaacagtcaacgtcggtggccagagctggacgctctactatggctacaacggagccatgcaagtctattcctttgtggcccagaccaacactaccaactacagcggagatgtcaagaacttcttcaattatctccgagacaataaaggatacaacgctgcaggccaatatgttcttagtaagtcaccctcactgtgactgggctgagtttgttgcaacgtttgctaacaaaaccttcgtataggctaccaatttggtaccgagcccttcacgggcagtggaactctgaacgtcgcatcctggaccgcatctatcaactaa

SEQ ID NO: 5 - protein, 222 aa, Trichoderma reesei
MVSFTSLLAASPPSRASCRPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 6 - protein, 227 aa, Humicola insolens
MVSLKSVLAAATAVSSAIAAPFDFVPRDNSTALQARQVTPNAEGWHNGYFYSWWSDGGGQVQYTNLEGSRYQVRWRNTGNFVGGKGWNPGTGRTINYGGYFNPQGNGYLAVYGWTRNPLVEYYVIESYGTYNPGSQAQYKGTFYTDGDQYDIFVSTRYNQPSIDGTRTFQQYWSIRKNKRVGGSVNMQNHFNAWQQHGMPLGQHYYQVVATEGYQSSGESDIYVQTH

SEQ ID NO: 7 - protein, 210 aa, Bacillus stearothermophilus
MKLKKKMLTLLLTASMSFGLFGATSSAATDYWQYWTDGGGMVNAVNGPGGNYSVTWQNTGNFVVGKGWTVGSPNRVINYNAGIWEPSGNGYLTLYGWTRNALIEYYVVDSWGTYRPTGNYKGTVNSDGGTYDIYTTMRYNAPSIDGTQTFQQFWSVRQSKRPTGSNVSITFSNHVNAWRSKGMNLGSSWAYQVLATEGYQSSGRSNVTVW

SEQ ID NO: 8 - protein, 229 aa, Trichoderma reesei
MVAFSSLICALTSIASTLAMPTGLEPESSVNVTERGMYDFVLGAHNDHRRRASINYDQNYQTGGQVSYSPSNTGFSVNWNTQDDFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 9 - protein, 211 aa, Aspergillus awamori
MKVTAAFAGLLVTAFAAPVPEPVLVSRSAGINYVQNYNGNLGDFTYDESAGTFSMYWEDGVSSDFVVGLGWTTGSSNAITYSAEYSASGSSSYLAVYGWVNYPQAEYYIVEDYGDYNPCSSATSLGTVYSDGSTYQVCTDTRTNEPSITGTSTFTQYFSVRESTRTSGTVTVANHFNFWAQHGFGNSDFNYQVMAVEAWSGAGSASVTISS

SEQ ID NO: 10 - protein, 330 aa, Bacillus stearothermophilus
MCSSIPSLREVFANDFRIGAAVNPVTLEAQQSLLIRHVNSLTAENHMKFEHLQPEEGRFTFDIAIKSSTSPFSSHGVRGHTLVWHNQTPSWVFQDSQGHFVGRDVLLERMKSHISTVVQRYKGKVYCWDVINEAVADEGSEWLRSSTWRQIIGDDFIQQAFLYAHEADPEALLFYNDYNECFPEKREKIYTLVKSLRDKGIPIHGIGMQAHWSLNRPTLDEIRAAIERYASLGVILHITELDISMFEFDDHRKDLAAPTNEMVERQAERYEQIFSLFKEYRDVIQNVTFWGIADDHTWLDHFPVQGRKNWPLLFDEQHNPKPAFWRVVNI

SEQ ID NO: 11 - protein, 190 aa, Trichoderma reesei
QTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 12 - protein, 237 aa, Aspergillus awamori
MKAFHLLAALSGAAVAQQAQLCDQYATYTGGVYTINNNLWGKDAGSGSQCTTVNSASSAGTSWSTKWNWSGGENSVKSYANSGLSFNKKLVSQISHIPTAARWSYDNTCIRRGRAYDLFTAADINHVTWSGDYELMIWLARYGGVQPLGSQIATATVEGQTWELWYGVNGAQKTYSFVAANPITSFQGDINDFFKYLTQNHGFPASSQYLITLQFGTEPFTGGPATLNVADWSASVQ

SEQ ID NO: 13 - protein, 233 aa, Trichoderma viride
MKFLQIAPTLLPVALAQSSCSQYATFSGGNYALSNNLWGQSAGSGSGCITDVSLGGSAVWSTTWDWSGGQSNVKGYPNIALNIPNKRLVSSISSMPTTAQWSYSGSSIRADVAYDLFTASNPNHVTYSGDYELMIWLGKYGDIQPIGSSQGTVNVGGTSWNLWYGPNGSMQVYSFVAPGNLTNWSGDVKNFYTYLQNNKGYPASSQYVLSYQFGTEAFTGSGTLNNTWTASIN

SEQ ID NO: 14 - protein, 234 aa, Hypocrea koningii
MKLIHVLPALIPAALAQTSCDQYAVFTGSDYTVSNNLWGQSAGSGFGCVTAESLSGSASWHADWQWSGGQNNVKSYQNSQIPIPQKRTVNSISSMPTTASWSYTGSDIRANVAYDLFTAANPNHVTYSGDYELMIWLGRYGDIGPIGSSQGTVNVGGQSWTLYYGYNGAMQVYSFVAQTNTTSYSGDVKNFFNYLRDNKGYNAAGQYVLSYQFGTEPFTGSGTLNVASWTASIN

SEQ ID NO: 15 - protein, 234 aa, Hypocrea schweinitzii
MKFLQVLPAILPAALAQTSCDQYATFSGNGYIVSNNLWGASAGSGFGCVTSVSLNGAASWHADWQWSGGQNNVKSYQNVQINIPQKRTVNSIGSMPTTASWSYSGSDIRANVAYDLFTAANPNHVTYSGDYELMIWLGKYGDIGPIGSSQGTVNVGGQTWTLYYGYNGAMQVYSFVAQSNTTSYSGDVKNFFNYLRDNKGYNAGGQYVLSYQFGTEPFTGSGTLNVASWTASIN

SEQ ID NO: 16 - protein, 237 aa, Stachybotrys echinata
MKVAALLVALSPLAFAQSLCDQYSYYSSNGYEFNNNMWGRNSGQGNQCTYVDYSSPNGVGWRVNWNWSGGDNNVKSYPYSGRQLPTKRIVSWIGSLPTTVSWNYQGNNLRANVAYDLFTAANPNHPNSSGDYELMIWLGRLGNVYPIGNQVATVNIAGQQWNLYYGYNGAMQVYSFVSPNQLNYFSGNVKDFFTYLQYNRAYPADSQYLITYQFGTEPFTGQNAVFTVSNWSAQQNN

SEQ ID NO: 17 - protein, 238 aa, Fusarium equiseti
MKSTLLLAGAFAPLAFAKDLCEQYGYLSSDGYSLNNNVWGKDSGTGDQCTHVNWNNANGAGWDVEWNWSGGKDNVKSYPNSALLIGEDKKTISSITNMQSTAEWKYSGDNLRADVAYDLFTAADPNHETSSGEYELMVWLARIGGVQPIGSLQTSVTIEGHTWELWVGMNGSMKVFSFVAPTPVNNFNADIKQFWDYLTKSQNFPADNQYLLTFQFGTEPFTGDNAKFTVTNFNAHLK

SEQ ID NO: 18 - protein, 237 aa, Bionectria ochroleuca
MKTGIAYLAAVLPLAMAESLCDQYAYLSRDGYNFNNNEWGAATGTGDQCTYVDSTSSGGVSWHSDWTNSGSESEIKSYPYSGLDLPEKKIVTSIGSISTGAEWSYSGSNIRADVAYDIFTAADPNHATSSGDYEVMIWLANLGGLTPIGSPIGTVKAAGRDWELWDGYNGAMRVYSFVAPSQLNSFDGEIMDFFYVVKDMRGFPADSQHLLTVQFGTEPISGSGAKFSVSHWSAKLG

SEQ ID NO: 19 - protein, 236 aa, Bionectria ochroleuca
MKFQLLSLTAFAPLSLAALCGQYQSQSQGGYIFNNNKWGQGSGSGSQCLTIDKTWDSNVAFHADWSWSGGTNNVKSYPNAGLEFSRGKKVSSIGTINGGADWDYSGSNIRANVAYDIFTSADPNHVTSSGDYELMIWLGKLGDIYPIGNSIGRVKAANREWDLHVGYNGAMKVFSFVAPSPVTRFDGNIMDFFYVMRDMQGYPMDKQYLLTLQFGTEPFTGSNAKFSCWYFGAKIK

SEQ ID NO: 20 - protein, 240 aa, Bionectria ochroleuca
MKANIVILSLFAPLAAVAQTLCGQYSSNTQGGYIFNNNMWGMGSGSGSQCTYVDKVWAEGVAWHTDWSWSGGDNNVKSYPYSGRELGTKRIVSSIKSISSGADWDYTGSNLRANAAYDIFTSANPNHATSSGDYEVMIWLGRYGGVYPIGNSIGTVRAAGRDWALHIGYNGAMKVFSFVAANPVTRFDGEIMDFFYLLRDMQGYPMTSQYLLTLQFGTEPFTGSGAKFNCWYFGATLSYW

SEQ ID NO: 21 - protein, 254 aa, Humicola grisea
MLKSALLLGAAAVSVQSASIPTIPANLEPRQIRSLCELYGYWSGNGYELLNNLWGKDTATSGWQCTYLDGTNNGGIQWNTAWEWQGAPDNVKNYPYVGKQIQRGRKISDINSMRTSVSWTYDRTDLRANVAYDVFTARDPDHPNWGGDYELMIWLARYGGIYPIGTFHSQVNLAGRTWDLWTGYNGNMRVYSFLPPSGDIRDFSCDIKDFFNYLERNHGYPAREQNLIVYQVGTECFTGGPARFTCRDFRADLW

SEQ ID NO: 22 - protein, 247 aa, Chaetomium brasiliense
MKLTLVLFVSSLAAATPLGWRERRQQVSLCGQSSSWSGNGYQLNNNLWGQSRATSGSQCTYLDSSSNSGIHWHTTWTWEGGEGEVKSYAYSGRQVSTGLTIASIDSMQTSVSWEYNTTDIQANVAYDIFTAEDPDHEHSSGDYEVMIWLARYNNVSPIGSSVATATVGGDTWDLFAGANGDMEVYSFVAENTMNSFSGDVKDFFDYLEQNVGFPVDDQYLLVFELGSEAFTGGPATLSVSQFSANIA

SEQ ID NO: 23 - protein, 357 aa, Bionectria ochroleuca
MKSIISFFGLATLVAAAPSQNPTRTQPLEKRATTLCGQWDSVETGGYTIYNNLWGQDNGSGSQCLTVEGVTDGLAAWSSTWSWSGGSSSVKSYSNAVLSAEAARISAISSIPSKWEWSYTGTDIVANVAYDLFSNTDCGDTPEYEIMIWLSALGGAGPISSTGSSIATVTIAGASWNLWQGQNNQMTVFSFVAESDQKSFSGDLNDFIQYLVDSQGYSGSQCLYSIGAGTEPFTGTDAEFITTGYSVSVSAGDSGSDETTTSSQAQSSTVETSTATQPQSSSTVVPTVTLSQPSNESTTTPVQSQPSSVETTPTAQPQSSSVQTTTTAQAQPTPERAAPDAGSAELLSSATMHLDRR

SEQ ID NO: 24 - protein, 247 aa, Emericella desertorum
MKLLALSLVSLASAASAASILSNTFTRRSDFCGQWDTATVGNFIVYNNLWGQDNADSGSQCTGVDSANGNSISWHTTWSWSGGSSSVKSYANAAYQFTSTKLNSLSSIPTSWKWQYSTTDIVANVAYDLFTSSSAGGDSEYEIMIWLAALGGAGPISSTGSSIATVTLGGVTWSLYSGPNGSMQVYSFVASSTTESFSADLMDFINYLAENQGLSSSQYLTHVQAGTEPFTGTDATLTVSSYSVSVS

SEQ ID NO: 25 - protein, 244 aa, Fusarium solani
MKSAIVAALAGLAAASPTRLIPRGQFCGQWDSETAGAYTIYNNLWGKDNAESGEQCTTNSGEQSDGSIAWSVEWSWTGGQGQVKSYPNAVVEIEKKTLGEVSSIPSAWDWTYTGNGIIANVAYDLFTSSTESGDAEYEFMIWLSALGGAGPISNDGSPVATVELAGTSWKLYQGKNNQMTVFSFVAESDVNNFCGDLADFTDYLVDNHGVSSSQILQSVGAGTEPFEGTNAVFTTNNYHADVEY

SEQ ID NO: 26 - protein, 250 aa, Fusarium solani
MKFFGVVSAFLAATAVATPTTPTETIEKRDTTWCDAFGSLATSGYTVYHNNWGKGDATSGSQCTTFTSVSNNNFVWSTSWTWAGGAGKVKSYSNVALEKINKKISDIKSVSTRWIWRYTGTKMIANVSYDLWFAPTASSNNAYEIMIWVGAYGGALPISTPGKGVIDRPTLAGIPWDVYKGPNGDVTVISFVASSNQGNFQADLKEFLNYLTSKQGLPSNYVATSFQAGTEPFEGTNAVLKTSAYTISVN

SEQ ID NO: 27 - protein, 371 aa, Streptomyces sp. 11AG8
MRSHPRSATMTVLVVLASLGALLTAAAPAQANQQICDRYGTTTIQDRYVVQNNRWGTSATQCINVTGNGFEITQADGSVPTNGAPKSYPSVYDGCHYGNCAPRTTLPMRISSIGSAPSSVSYRYTGNGVYNAAYDIWLDPTPRTNGVNRTEIMIWFNRVGPVQPIGSPVGTAHVGGRSWEVWTGSNGSNDVISFLAPSAISSWSFDVKDFVDQAVSHGLATPDWYLTSIQAGFEPWEGGTGLAVNSFSSAVNAGGGNGGTPGTPAACQVSYSTHTWPGGFTVDTTITNTGSTPVDGWELDFTLPAGHTVTSVWNALISPASGAVTARSTGSNGRIAANGGTQSFGFQGTSSGAGFTAPAGARLNGTSCTVR

SEQ ID NO: 28 - protein, 221 aa, Artificial Sequence
  consensus sequence
  VARIANT (1)...(221): Xaa = Any Amino Acid
CXQYXXXXXXGYXXXNNXWGXXXXXSGXQCTXXXXXXXXXXXXWXXXWXWSGGXXXVKSYXXXXXXXXXXXXXXSXIXSXXXXXXWXYXGXXXXANVAYDLFTXXXPXHXXXXGXYEXMIWLXXXGGXXPIGSXXXXVXXXXXXGXXWXLXXGXNGXMXVXSFVAXSSSSSSFXGDXXXFXXYLXXXXGXPXXXQYLXXXQXGTEPFTGXXAXXXXXXXXA

SEQ ID NO: 29 - DNA, 25 bp, Artificial Sequence
  synthetic oligonucleotide
gaacgatggcaagggcggcgtgacg

SEQ ID NO: 30 - DNA, 27 bp, Artificial Sequence
  synthetic oligonucleotide
cttctcgggctgctacaacccaaacgg

SEQ ID NO: 31 - DNA, 26 bp, Artificial Sequence
  synthetic oligonucleotide
acatcgtcgagtgttttggcacctac

SEQ ID NO: 32 - DNA, 29 bp, Artificial Sequence
  synthetic oligonucleotide
catcgtcgagaactggggcacctacaacc

SEQ ID NO: 33 - DNA, 21 bp, Artificial Sequence
  synthetic oligonucleotide
ggcacctaccgaccgtccacg

SEQ ID NO: 34 - DNA, 25 bp, Artificial Sequence
  synthetic oligonucleotide
caagctgggcgagcacacctccgac

SEQ ID NO: 35 - DNA, 21 bp, Artificial Sequence
  synthetic oligonucleotide
cgccgcaactgtcgctcgagc

SEQ ID NO: 36 - DNA, 29 bp, Artificial Sequence
  synthetic oligonucleotide
gtggagggttaccaaagctctggctctgc

SEQ ID NO: 37 - DNA, 27 bp, Artificial Sequence
  synthetic oligonucleotide
tctggctctgcttgcatcaccgtcagc

SEQ ID NO: 38 - DNA, 27 bp, Artificial Sequence
  synthetic oligonucleotide
gagaagcgccagtgcattcagcccggc

SEQ ID NO: 39 - DNA, 27 bp, Artificial Sequence
  synthetic oligonucleotide
gtgacgtactgcaatggtcccggcggg

SEQ ID NO: 40 - DNA, 33 bp, Artificial Sequence
  synthetic oligonucleotide
ggcaccaagaacagggtcatcaacttctcgggc

SEQ ID NO: 41 - DNA, 33 bp, Artificial Sequence
  synthetic oligonucleotide
tccatcaccgtcagcgattaaagggggctcttc

SEQ ID NO: 42 - DNA, 32 bp, Artificial Sequence
  synthetic oligonucleotide
cccagacgattcagtgcggcacgggctacaac

SEQ ID NO: 43 - DNA, 32 bp, Artificial Sequence
  synthetic oligonucleotide
cttctactcgtactggtgcgatggccacggcg

SEQ ID NO: 44 - DNA, 32 bp, Artificial Sequence
  synthetic oligonucleotide
cgattcagcccggctgcggctacaacaacggc

SEQ ID NO: 45 - DNA, 35 bp, Artificial Sequence
  synthetic oligonucleotide
caacggctacttctactgctactggaacgatggcc

SEQ ID NO: 46 - DNA, 34 bp, Artificial Sequence
  synthetic oligonucleotide
ccggcacgggctactgcaacggctacttctactc

SEQ ID NO: 47 - DNA, 31 bp, Artificial Sequence
  synthetic oligonucleotide
ggcgtgacgtacacctgcggtcccggcgggc

SEQ ID NO: 48 - DNA, 27 bp, Artificial Sequence
  synthetic oligonucleotide
ggcgccaccaagtgcggcgaggtcacc

SEQ ID NO: 49 - DNA, 28 bp, Artificial Sequence
  synthetic oligonucleotide
gcgtgggctcagtgcggcctgacgctcg

SEQ ID NO: 50 - DNA, 752 bp, Trichoderma reesei
atggttgccttttccagcctcatctgcgctctcaccagcatcgccagtactctggcgatgcccacaggcctcgagcctgagagcagtgtcaacgtcacagagcgtggcatgtacgactttgttcttggagctcacaatgatcatcgccgtcgtgctagcatcaactacgaccaaaactaccaaactggcggacaagtcagctattcgccttccaacactggcttctcagtgaactggaacactcaagatgactttgttgtgggcgttggttggacgactggatcttctgcgtaggaggactcctcatcattctgcactttgaaagcatcttctgaccaaaagcttctcttagtcccatcaactttggcggctcttttagtgtcaacagcggaactggcctgctttccgtctatggctggagcaccaacccactggttgagtactacatcatggaggacaaccacaactacccagcacagggtaccgtcaagggaaccgtcaccagcgacggagccacttacaccatctgggagaatacccgtgtcaacgagccttccatccagggcacagcgaccttcaaccagtacatttccgtgcggaactcgcccaggaccagcggaactgttactgtgcagaaccacttcaatgcttgggcctcgcttggcctgcaccttgggcagatgaactaccaggttgtcgctgtcgaaggctggggtggtagtggttctgcctcacagagtgtcagcaactag

SEQ ID NO: 51 - protein, 248 aa, Trichoderma reesei
MVAFSSLICALTSIASTLAMPTGLEPESSVNVTERGMYDFVLGAHNDHRRRASINYDQNYQTGGQVSYSPSNTGFSVNWNTQDDFVVGVGWTTGSSAEDSSSFCTLKASSDQKLLLVPSTLAALLVSTAELACFPSMAGAPTHWLSTTSWRTTTTTQHRVPSREPSPATEPLTPSGRIPVSTSLPSRAQRPSTSTFPCGTRPGPAELLLCRTTSMLGPRLACTLGRTTRLSLSKAGVVVVLPHRVSAT